(19) United States
(12) Patent Application Publication
(10) Pub. No.: US 2025/0282695 A1
(43) Pub. Date: Sep. 11, 2025

(54) GALLIUM ZEOLITES AND METHODS OF USE THEREOF

(71) Applicant: UNIVERSITY OF HOUSTON SYSTEM, Houston, TX (US)

(72) Inventors: Jeffrey D. Rimer, Bellaire, TX (US); Amir Abutalib, Houston, TX (US); Deependra Parmar, Houston, TX (US)

(21) Appl. No.: 18/600,236

(22) Filed: Mar. 8, 2024

(51) Int. Cl. C07C 1/24; B01J 8/02; B01J 29/40; B01J 29/70

(52) U.S. Cl. CPC C07C 1/24; B01J 8/0278; B01J 29/405; B01J 29/7065; B01J 29/7088; C07C 2529/40

(57) ABSTRACT

This invention relates to gallium zeolites. This invention also relates to systems and methods utilizing the gallium zeolites.

### STATEMENT REGARDING FEDERALLY SPONSORED RESEARCH OR DEVELOPMENT

This invention was made with government support under Grant No. DE-SC0014468 awarded by the U.S. Department of Energy. The government has certain rights in the invention.

### FIELD OF INVENTION

This invention relates to gallium zeolites. This invention also relates to systems and methods utilizing the gallium zeolites.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Light olefins are heavily utilized in commercial processes as fundamental building blocks for a myriad of materials that include plastics, synthetic fibers, packaging materials, and automotive parts. The market demand for light olefins, notably ethylene and propylene, is expected to grow to around 165 million tons by 2030. The most common route of light olefin production is the thermal cracking of naphtha, which is considered the single-most energy consuming process in the chemical industry.

Less energy intensive routes of olefin production include reactions such as methanol to olefins (MTO), alkane dehydrogenation, synthesis gas to olefins, and alcohol dehydration. In particular, alcohol dehydration reactions offer an interesting pathway to olefin production as they are derived from cheap, readily-available alcohol feedstocks. As such, there is an ongoing need for improvements in materials and methods for alcohol dehydration reactions and olefin production reactions. The embodiments of the present invention address that need.

### SUMMARY OF THE INVENTION

In various embodiments, the present invention provides a method for the dehydration of at least one alcohol, the method comprising: contacting a feedstock comprising at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms. In some embodiments, contacting the feedstock comprising at least one alcohol with the at least one zeolite is performed at a temperature of 180° C. to 600° C. In some embodiments, contacting the feedstock comprising at least one alcohol with the at least one zeolite is performed at a weight-hourly space velocity (WHSV) of 2 h⁻¹ to 20 h⁻¹. In some embodiments, the at least one zeolite comprises at least one extra-framework species, with the proviso that the at least one extra-framework species does not comprise aluminum. In some embodiments, the at least one extra-framework species is Ga₂O₃. In some embodiments, the framework type is MWW, CHA, or MFI. In some embodiments, the at least one zeolite is Ga-MCM-22 zeolite, Ga-SSZ-13 zeolite, or Ga-ZSM-5 zeolite. In some embodiments, the at least one zeolite is Ga-MCM-22 zeolite and the first framework type is MWW. In some embodiments, the at least one zeolite is a Ga-SSZ-13 zeolite and the first framework type is CHA. In some embodiments, the at least one zeolite is a Ga-ZSM-5 zeolite and the first framework type is MFI. In some embodiments, the at least one alcohol is selected from the group consisting of methanol, ethanol, and combination thereof. In some embodiments, the at least one product is at least one hydrocarbon, dimethyl ether, water, or any combination thereof. In some embodiments, the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, and any combination thereof. In some embodiments, the at least one hydrocarbon is at least one olefin, or at least one aromatic hydrocarbon, or both at least one olefin and at least one aromatic hydrocarbon. In some embodiments, the at least one olefin is selected from the group consisting of ethylene, propylene, and combination thereof.

In various embodiments, the present invention provides a system for the dehydration of at least one alcohol, comprising: an inlet port; a reaction chamber, wherein the reaction chamber is in communication with the inlet port, wherein the reaction chamber contains at least one zeolite, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms; and an outlet port, wherein the outlet port is in communication with the reaction chamber. In some embodiments, the at least one zeolite comprises at least one extra-framework species, with the proviso that the at least one extra-framework species does not comprise aluminum. In some embodiments, the framework type is MWW, CHA, or MFI. In some embodiments, the at least one zeolite is Ga-MCM-22 zeolite, Ga-SSZ-13 zeolite, or Ga-ZSM-S zeolite. In some embodiments, the at least one zeolite is Ga-MCM-22 zeolite and the framework type is MWW, or wherein the at least one zeolite is a Ga-SSZ-13 zeolite and the framework type is CHA, or wherein the at least one zeolite is a Ga-ZSM-5 zeolite and the first framework type is MFI.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts in accordance with various embodiments of the invention, visual representation of the dual bed reactor: Ga-zeolite (upstream bed B1) and Al-zeolite (downstream bed B2) with an alcohol feed (methanol, ethanol), key intermediates (DME, ethylene), and olefin products (ethylene, propylene).

FIG. 2A-FIG. 2F depicts in accordance with various embodiments of the invention, scanning electron microscopy (SEM) images of proton-form (FIG. 2A) Al-MCM-22, (FIG. 2B) Ga-MCM-22, (FIG. 2C) Al-SSZ-13, (FIG. 2D) Ga-SSZ-13, (FIG. 2E) Al-ZSM-5, and (FIG. 2F) Ga-ZSM-5.

FIG. 3A-FIG. 3C depicts in accordance with various embodiments of the invention, XPS spectra of Ga 2p for (FIG. 3A) Ga-MCM-22, (FIG. 3B) Ga-SSZ-13, and (FIG. 3C) Ga-ZSM-5 proton-form samples. Spectra have been corrected using carbon peaks as a reference. Peak deconvolution was performed using Gaussian fits to extract relative contributions of framework (thick dashed line) and extra-framework (thick solid line) gallium species from the sum of both contributions (thin dashed line). The thin solid line is the originally acquired signal from the XPS instrument before being fitted by the sum of both contributions (thin dashed line).

FIG. 4A-FIG. 4G depicts in accordance with various embodiments of the invention, (FIG. 4A) Solid-state MAS ⁷¹Ga NMR spectra of Ga-ZSM-5 (top), Ga-SSZ-13 (middle), and Ga-MCM-22 (bottom) proton-form samples. (FIG. 4B-FIG. 4G) are potential framework and extra-framework Ga species that have been reported in the literature for Ga-zeolites (Zhou, Y.; Thirumalai, H.; Smith, S. K.; Whitmire, K. H.; Liu, J.; Frenkel, A. I., Grabow, L. C.; Rimer, J. D., Ethylene dehydroaromatization over Ga-ZSM-5 catalysts: nature and role of gallium speciation. Angew. Chem. Int. Ed. 2020, 59 (44), 19592-19601; Phadke, N. M.; Mansoor, E.; Bondil, M.; Head-Gordon, M.; Bell, A. T., Mechanism and kinetics of propane dehydrogenation and cracking over Ga/H-MFI prepared via vapor-phase exchange of H-MFI with GaCl3. J. Am. Chem. Soc. 2018, 141 (4), 1614-1627).

FIG. 5A-FIG. 5B depicts in accordance with various embodiments of the invention, comparison of Ga-zeolites for methanol dehydration at 350° C. and a weight-hourly space velocity (WHSV) of 8 h⁻¹. (FIG. 5A) Conversion of methanol over proton-form Ga-MCM-22, Ga-SSZ-13, and Ga-ZSM-5 catalysts as a function of time on stream (TOS). (FIG. 5B) Corresponding product selectivity of each Ga-zeolite. A full comparison of product selectivity as a function of TOS is provided in FIG. 13A-FIG. 13B. Solid lines are interpolated to guide the eye.

FIG. 6 depicts in accordance with various embodiments of the invention, comparison of methanol dehydration catalysts reported in literature with Ga-MCM-22 (star) of the present invention. Details of each catalyst and corresponding reaction conditions are provided in Table 2. Data were obtained from the review of Bateni and Able (Bateni, H.; Able, C., Development of heterogeneous catalysts for dehydration of methanol to dimethyl ether: A review. Catal. Ind. 2019, 11, 7-33) with the omission of several classes of methanol dehydration catalysts that are outside the compositional space of materials examined in the work disclosed herein (e.g., heteropoly acids, metal oxides, ion-exchange resins, and quasicrystals).

FIG. 7A-FIG. 7B depicts in accordance with various embodiments of the invention, (FIG. 7A) Methanol/DME conversion during the methanol-to-hydrocarbons reaction over dual catalyst beds (B1 and B2 in FIG. 1) with the following combinations: (i) B1=Ga-MCM-22 and B2=Al-ZSM-5 (triangles); (ii) B1=Ga-SSZ-13 and B2=Al-ZSM-5 (squares); and (iii) B1=Ga-ZSM-5 and B2=Al-ZSM-5 (hexagons). Reactions were carried out at 350° C. with WHSV=7 and 3 h⁻¹ for upstream and downstream beds, respectively. Comparisons were made to single catalyst beds containing Al-ZSM-5 (control, circles) and a physical mixture of Ga-MCM-22 and Al-ZSM-5 (diamonds). Solid lines are interpolated to guide the eye. (FIG. 7B) corresponding product selectivities evaluated at the initial time on stream (ca. 30 min) for both dual and single beds.

FIG. 8A-FIG. 8B depicts in accordance with various embodiments of the invention, comparison of Ga-zeolites for ethanol dehydration at 500° C. and WHSV=19 h⁻¹. (FIG. 8A) Conversion of ethanol over proton-form Ga-MCM-22, Ga-SSZ-13, and Ga-ZSM-5 as a function of time on stream (TOS). (FIG. 8B) Corresponding ethylene selectivity of each Ga-zeolite. A full comparison of product selectivity as a function of TOS is provided in FIG. 16A-FIG. 16B. Solid lines are interpolated to guide the eye.

FIG. 9A-FIG. 9D depicts in accordance with various embodiments of the invention, production of propylene from ethanol at 500° C. and WHSV=5 and 3 h⁻¹ for upstream and downstream beds, respectively. Four bed configurations were tested: (FIG. 9A) single bed of Al-SSZ-13, (FIG. 9B) a dual bed with tandem catalysts Ga-MCM-22 (B1) and Al-SSZ-13 (B2), (FIG. 9C) a dual bed with Ga-SSZ-13 (B1) and Al-SSZ-13 (B2), and (FIG. 9D) a dual bed with Ga-ZSM-5 (B1) and Al-SSZ-13 (B2). For each set of data, the conversion (circles) is the left y-axis and the selectivity (squares, triangles, hexagons, and diamonds) is the right y-axis. Solid lines are interpolated to guide the eye.

FIG. 10 depicts in accordance with various embodiments of the invention, space time yield (STY) of propylene for the conversion of ethanol to propylene using the following fixed bed reactor configurations: squares, Ga-MCM-22(B1)-Al-SSZ-13(B2) dual bed; triangles, Ga-SSZ-13(B1)-Al-SSZ-13(B2) dual bed; diamonds, Ga-ZSM-5(B1)-Al-SSZ-13(B2) dual bed; circles, Al-SSZ-13 single bed. Solid lines are interpolated to guide the eye.

FIG. 11 depicts in accordance with various embodiments of the invention, powder X-ray diffraction (XRD) patterns of commercially-received Ga₂O₃ (reference) and as-synthesized Ga- and Al-zeolites used in the work described herein.

FIG. 12 depicts in accordance with various embodiments of the invention, N₂ adsorption/desorption isotherms of as-synthesized Ga-zeolites: Ga-MCM-22 (squares), Ga-ZSM-5 (circles), and Ga-SSZ-13 (triangles).

FIG. 13A-FIG. 13B depicts in accordance with various embodiments of the invention, methanol (MeOH) conversion (squares, left axes) and product selectivity (right axes) of (FIG. 13A) Ga-SSZ-13 and (FIG. 13B) Ga-ZSM-5 corresponding to data presented in FIG. 5A-FIG. 5B. Solid lines are interpolations to guide the eye.

FIG. 14 depicts in accordance with various embodiments of the invention, Methanol (MeOH) conversion and product selectivity for the Ga-MCM-22 catalyst during methanol dehydration at 250° C.

FIG. 15 depicts in accordance with various embodiments of the invention, methanol (MeOH) conversion (squares) and DME product selectivity (circles) for commercially-received Ga₂O₃ during methanol dehydration at 350° C. and WHSV=8 h⁻¹.

FIG. 16A-FIG. 16B depicts in accordance with various embodiments of the invention, ethanol (EtOH) conversion (squares, left axes) and product selectivity (right axes) of (FIG. 16A) Ga-SSZ-13 and (FIG. 16B) Ga-ZSM-5 corresponding to data presented in FIG. 8A-FIG. 8B. Solid lines are interpolations to guide the eye.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For purposes of the present invention, the following terms are defined below. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, apparatus, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

In some embodiments, the numbers expressing quantities of reagents, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

The term “Cn” hydrocarbon means hydrocarbon having n carbon atom(s) per molecule, where n is a positive integer. In some embodiments, n is 1-20. In some embodiments. n is 1-10. In some embodiments, n is 1-5.

The term “C₁” hydrocarbon means hydrocarbon having 2 carbon atoms per molecule.

The term “C₂” hydrocarbon means hydrocarbon having 2 carbon atoms per molecule.

The term “C₃” hydrocarbon means hydrocarbon having 3 carbon atoms per molecule.

The term “C₄” hydrocarbon means hydrocarbon having 4 carbon atoms per molecule.

The term “C₅” hydrocarbon means hydrocarbon having 5 carbon atoms per molecule.

The term “C₆” hydrocarbon means hydrocarbon having 6 carbon atoms per molecule.

The term “C₇” hydrocarbon means hydrocarbon having 7 carbon atoms per molecule.

The term “C₈” hydrocarbon means hydrocarbon having 8 carbon atoms per molecule.

The term “hydrocarbon” means a class of compounds containing hydrogen bound to carbon, and encompasses (i) saturated hydrocarbon, (ii) unsaturated hydrocarbon, and (iii) mixtures of hydrocarbons, and including mixtures of hydrocarbon compounds (saturated and/or unsaturated), including mixtures of hydrocarbon compounds having different values of n.

The terms “olefin” and “olefinic hydrocarbon” refer to one or more unsaturated hydrocarbon compound containing at least one carbon atom directly bound to another carbon atom by a double bond. In other words, an olefin is a compound which contains at least one pair of carbon atoms, where the first and second carbon atoms of the pair are directly linked by a double bond. An olefin having a particular number of carbon atoms is the “corresponding olefinic hydrocarbon” of paraffinic hydrocarbon having the same number of carbon atoms. For example, olefinic C₄ hydrocarbon (normal butenes and/or isobutene) is the corresponding olefinic hydrocarbon of paraffinic C₄ hydrocarbon (butane and/or isobutane).

The term “aromatics” and “aromatic hydrocarbon” mean hydrocarbon compounds containing at least one aromatic core.

The term “selectivity” refers to the production of a specified compound in a catalytic reaction. As an example, the phrase “methanol conversion reaction has a 100% selectivity for dimethyl ether (DME)” means that 100% of the methanol that is converted in the reaction is converted to dimethyl ether (DME). When used in connection with a specified reactant, the term “conversion” means the amount of the reactant consumed in the reaction. For example, when the specified reactant is methanol, 100% conversion means 100% of the methanol is consumed in the reaction. Yield is conversion times selectivity.

The term “alcohol” means any compound substituted with an —OH group.

In some embodiments, the zeolite may be a naturally occurring zeolite or a synthetically manufactured zeolite. In some embodiments the second zeolite may be a naturally occurring zeolite or a synthetically manufactured zeolite. In some embodiments, the zeolite comprises a synthetic zeolite. In some embodiments, the zeolite is a synthetic zeolite. In some embodiments, the zeolite comprises a naturally occurring zeolite. In some embodiments, the zeolite is a naturally occurring zeolite. In some embodiments, the zeolite does not comprise a naturally occurring zeolite. In some embodiments, the zeolite is not a naturally occurring zeolite. In some embodiments, the zeolite of the present invention is not a naturally occurring zeolite. In some embodiments, the zeolite of the present invention does not comprise a naturally occurring zeolite. In some embodiments, the zeolite of the present invention comprises a synthetic zeolite. In some embodiments, the zeolite of the present invention is a synthetic zeolite. In some embodiments, the gallium zeolite is a synthetic zeolite. In some embodiments, the gallosilicate zeolite is a synthetic zeolite. In some embodiments, the gallium zeolite is not a naturally occurring zeolite. In some embodiments, the gallosilicate zeolite is not a naturally occurring zeolite. In some embodiments, the gallium zeolite comprises a synthetic zeolite. In some embodiments, the gallosilicate zeolite comprises a synthetic zeolite. In some embodiments, the aluminum zeolite is a synthetic zeolite. In some embodiments, the aluminum zeolite comprises a synthetic zeolite. In some embodiments, the aluminosilicate zeolite is a synthetic zeolite. In some embodiments, the aluminosilicate zeolite comprises a synthetic zeolite. In some embodiments, the aluminum zeolite does not comprise a naturally occurring zeolite. In some embodiments, the aluminum zeolite is not a naturally occurring zeolite. In some embodiments, the aluminosilicate zeolite is not a naturally occurring zeolite. In some embodiments, the aluminosilicate zeolite does not comprise a naturally occurring zeolite. In some embodiments, the first zeolite comprises a synthetic zeolite. In some embodiments, the first zeolite is a synthetic zeolite. In some embodiments, the first zeolite is not a naturally occurring zeolite. In some embodiments, the first zeolite does not comprise a naturally occurring zeolite. In some embodiments, the second zeolite is a synthetic zeolite. In some embodiments, the second zeolite comprises a synthetic zeolite. In some embodiments, the second zeolite is not a naturally occurring zeolite. In some embodiments, the second zeolite does not comprise a naturally occurring zeolite. In some embodiments, the at least one zeolite is a synthetic zeolite. In some embodiments, the at least one zeolite comprises a synthetic zeolite. In some embodiments, the at least one zeolite is not a naturally occurring zeolite. In some embodiments, the at least one zeolite does not comprise a naturally occurring zeolite.

In various embodiments, compounds (e.g., zeolites) of the present invention as disclosed herein may be synthesized using any synthetic method available to one of skill in the art. Non-limiting examples of synthetic methods used to prepare various embodiments of compounds of the present invention (e.g., zeolites of the present invention) are disclosed in the Examples section herein.

Zeolites are versatile catalysts owing to their tunable acidity where the active site(s) can be placed within the crystal framework or as extra-framework species in confined pores. In the work disclosed herein, we prepare a series of three different zeolites with MWW, CHA, and MFI frameworks as aluminosilicates and gallosilicates to demonstrate their performance in alcohol dehydration reactions. Each three-letter code corresponds to the crystal structure assigned by the International Zeolite Association. The two reactions examined in the work disclosed herein are methanol conversion to dimethyl ether and ethanol conversion to ethylene. Our findings reveal that Ga-zeolites exhibit superior performance with Ga-MCM-22 (MWW) achieving nearly 100% alcohol conversion and selectivity to desired products at contact times that are significantly less than most dehydration catalysts reported in the literature. The unique properties of the Ga-zeolites of the present invention are attributed to their reduced acidity via a direct (one-pot) synthesis that avoids conventional time-intensive, multi-step post-synthesis modifications to reduce the acid site density of Al-zeolites. Herein, in various embodiments of the present invention, we demonstrate the use of Ga-zeolites as tandem catalysts when paired with a downstream Al-zeolite in a dual-bed reactor configuration to convert cheaper reagents (alcohols) upstream to more desired intermediates as feeds for downstream catalysts to produce light olefins. We demonstrate that dual beds using Ga-zeolite-Al-ZSM-5 (upstream-downstream) pairings outperform single bed configurations with Al-ZSM-5 or a physical mixture (Ga-/Al-zeolite) for methanol to hydrocarbons reactions, with notable improvements in catalyst lifetime and increased light olefins selectivity. Similarly, dual beds using Ga-zeolite-Al-SSZ-13 (upstream-downstream) pairings result in improved performance for ethanol to propylene reactions with significant increases in propylene selectivity (>20%) compared to a conventional single bed configuration of Al-SSZ-13. Overall, this study offers a new perspective for the use of heteroatom-exchanged zeolite catalysts in tandem reactions as a means of capitalizing on their reduced acidity.

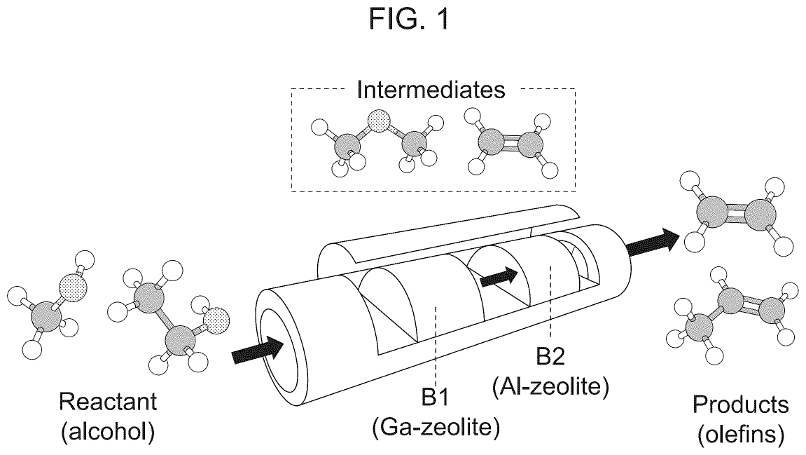

In the work disclosed herein, we capitalize on the ability to tune the acidity of heteroatom-substituted zeolites in a dual-bed reactor configuration (FIG. 1) where a less acidic Ga-zeolite is used in the upstream bed to convert alcohols to desirable intermediates prior to their conversion to olefins in a downstream bed containing a more acidic Al-zeolite. Herein we disclose one-pot syntheses of three gallosilicates (Ga-zeolites) with MCM-22 (MWW), SSZ-13 (CHA), and ZSM-5 (MFI) structures. Our findings reveal that Ga-zeolites are both active and selective alcohol dehydration catalysts, which are paired with commercially-relevant Al-zeolites (ZSM-5 and SSZ-13) in two different tandem configurations to upgrade the products of methanol and ethanol dehydration, respectively.

### Preparation of Zeolite Catalysts.

We prepared a series of gallosilicates (Ga-zeolites) along with their corresponding aluminosilicates (Al-zeolites) with three different crystal structures: CHA (SSZ-13), MFI (ZSM-5), and MWW (MCM-22). The preparation of Ga-ZSM-5 (Zhou, Y.; Thirumalai, H.; Smith, S. K.; Whitmire, K. H.; Liu, J., Frenkel, A. I.; Grabow, L. C.; Rimer, J. D., Ethylene dehydroaromatization over Ga-ZSM-5 catalysts: nature and role of gallium speciation. Angew. Chem. Int. Ed. 2020, 59 (44), 19592-19601) and all Al-zeolites (Parmar, D.; Cha, S. H., Huang, C.; Chiang, H.; Washburn, S.; Grabow, L. C.; Rimer, J. D., Impact of medium-pore zeolite topology on para-xylene production from toluene alkylation with methanol. Catal. Sci. Technol. 2023, 13 (18), 5227-5236; Perez-Uriarte, P.; Ateka, A.; Gamero, M.; Aguayo, A. T., Bilbao, J., Effect of the Operating Conditions in the Transformation of DME to olefins over a HZSM-5 Zeolite Catalyst. Ind. Eng. Chem. Res. 2016, 55 (23), 6569-6578) were performed using established protocols, whereas the synthesis of Ga-MCM-22 and Ga-SSZ-13 required modifications to reported synthesis protocols.

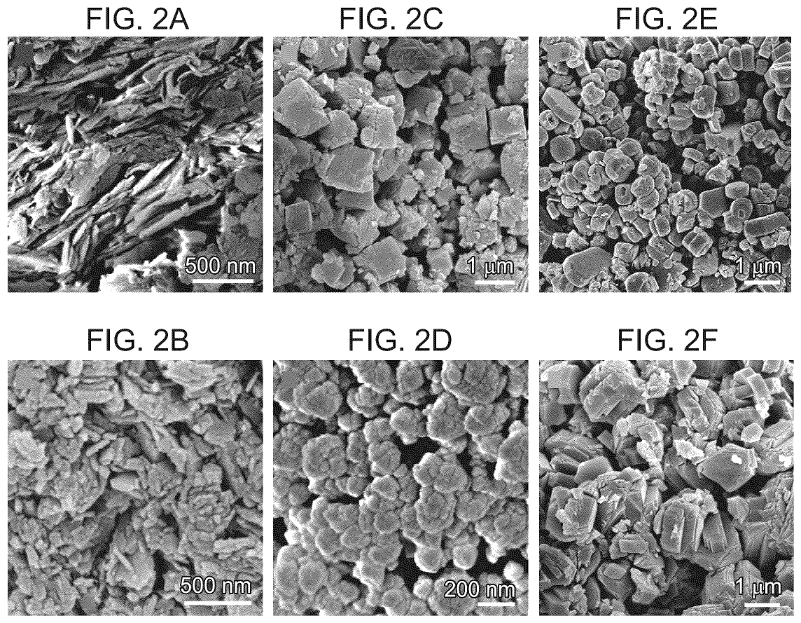

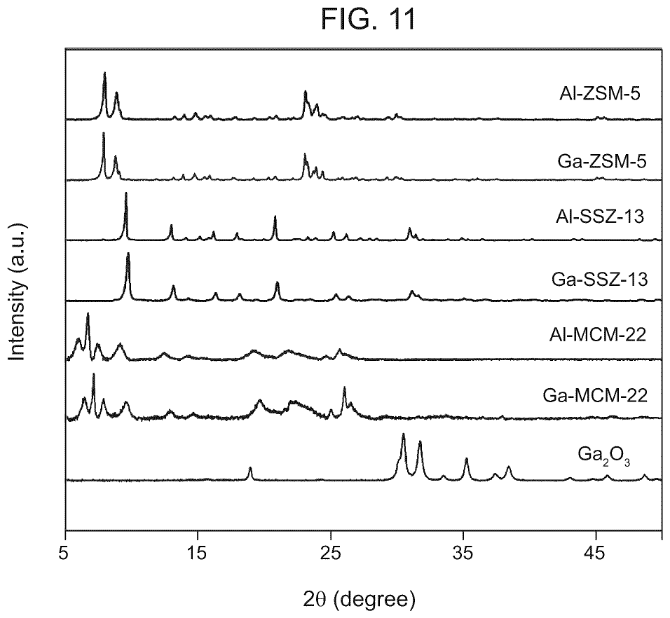

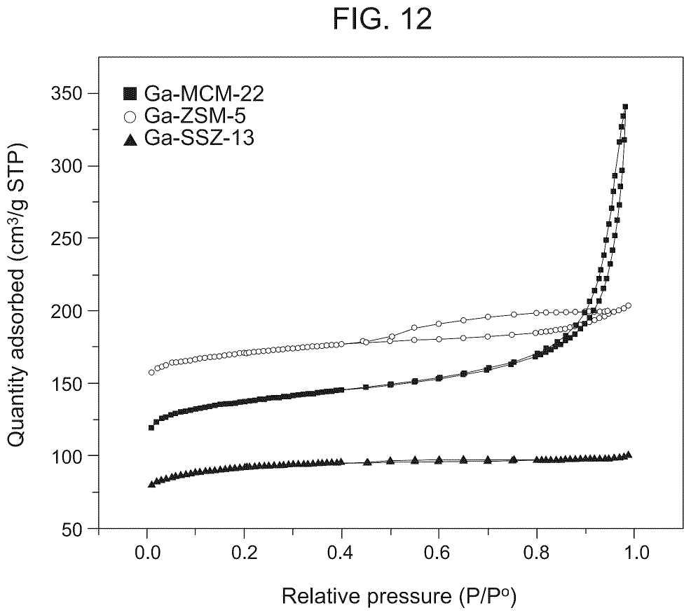

Powder X-ray diffraction (PXRD) of all as-synthesized materials revealed fully-crystalline products without observation of zeolite impurities (FIG. 11). Only the Ga-MCM-22 sample contained small peaks in its PXRD pattern that are not observed for Al-MCM-22, suggesting the presence of trace extra-framework gallium oxide based on similar peaks observed for commercial Ga₂O₃ (in the region 2θ=35−50°. Scanning electron micrographs (FIG. 2A, FIG. 2C, FIG. 2E) revealed the morphology of aluminosilicates Al-MCM-22, Al-SSZ-13, and Al-ZSM-5 as being layered, cubic, and coffin-shaped, respectively. For gallosilicate isostructures, the replacement of Al with Ga did not have a significant effect on crystal size (with the exception of SSZ-13). Analysis of extracted solids by energy dispersive X-ray (EDX) spectroscopy before and after synthesis revealed little difference in the Si/M ratio (M=Ga or Al) between the initial (amorphous) gel and final crystalline product (Table 1). Textural analysis of as-synthesized zeolite samples (Table 1) using nitrogen adsorption/desorption isotherms (FIG. 12) provided the total BET surface area, external surface area, and micropore volume (V_{micro}). We observed a slight reduction in total surface area when Al was replaced by Ga, suggesting a higher percentage of extra-framework species in Ga-zeolites. The acidity of H-form zeolites (Table 1) was extracted using a combination of NH₃ temperature-programmed desorption and pyridine FTIR measurements to quantify the total acidity and relative Brønsted/Lewis acidity, respectively.

TABLE 1
Physiochemical properties of Ga- and Al-zeolites.

| | | | BET surface | | | | | |
|---|---|---|---|---|---|---|---|---|
| | Si/M | | area ^{b} (m²/g) | | | | | |
| | ratio ^{a} | | Total | External | V_{micro}^{b} | | | |
| Zeolite | | Pro- | surface | surface | (cm³/ | Acidity (μmol g⁻¹) | | |
| catalysts | Gel | duct | area | area | g) | Total ^{c} | Brønsted ^{d} | Lewis ^{d} |
| Ga-MCM- | 15 | 15 | 513 | 109 | 0.17 | 491 | 226 | 265 |
| 22 | | | | | | | | |
| Ga-SSZ-13 | 15 | 15 | 633 | 88 | 0.23 | 352 | 281* | 71* |
| Ga-ZSM-5 | 15 | 17 | 344 | 71 | 0.11 | 633 | 506 | 127 |
| Al-MCM- | 15 | 15 | 634 | 149 | 0.19 | 485 | 412 | 73 |
| 22 | | | | | | | | |
| Al-SSZ-13 | 15 | 20 | 655 | 60 | 0.24 | 666 | 612* | 54* |
| Al-ZSM-5 | 50 | 55 | 385 | 97 | 0.11 | 441 | 370 | 71 |

ª Determined by elemental analysis (EDX) where M = Ga or Al;
^{b} N₂ adsorption/desorption data;
^{c} Determined by NH₃ TPD;
^{d} Determined by multiplying the total acidity by percentages obtained from FTIR pyridine adsorption;
*FTIR measurements of small-pore zeolites using pyridine (bulky molecule) only probes external sites; thus, values listed here do not reflect bulk properties.

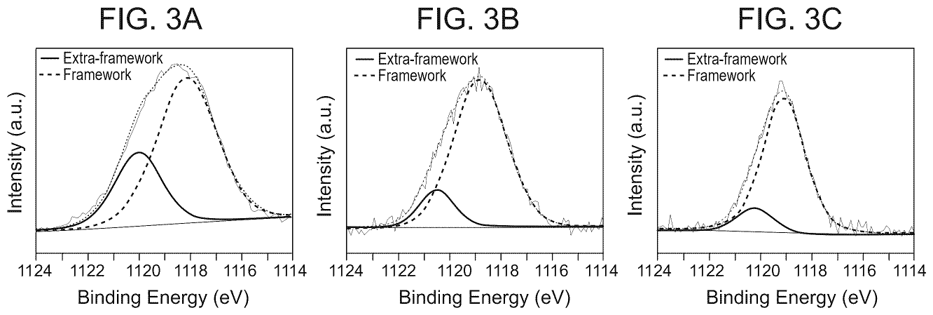

X-ray photoelectron spectroscopy (XPS) was used to characterize gallium speciation in each Ga-zeolite (FIG. 3A-FIG. 3C). The Ga 2p XPS spectra were fitted by two peaks corresponding to framework (1118-1119 eV) and extra-framework (1119.5-1120.5 eV) gallium, respectively (Oozeerally, R.; Pillier, J.; Kilic, E.; Thompson, P. B.; Walker, M.; Griffith, B. E.; Hanna, J. V.; Degirmenci, V., Gallium and tin exchanged Y zeolites for glucose isomerisation and 5-hydroxymethyl furfural production. Appl. Catal. A-Gen 2020, 605, 117798; Xin, M.; Xing, E., Gao, X.; Wang, Y.; Ouyang, Y.; Xu, G., Luo, Y.; Shu, X., Ga substitution during modification of ZSM-5 and its influences on catalytic aromatization performance. Ind. Eng. Chem. Res. 2019, 58 (17), 6970-6981). The Ga-MCM-22 sample contained the highest quantity (29%, FIG. 3A) of extra-framework Ga species, consistent with its large reduction in total surface area compared to Al-MCM-22. The quantity of extra-framework Ga species was significantly less for Ga-SSZ-13 (15%, FIG. 3B) and Ga-ZSM-5 (11%, FIG. 3B). The presence of extra-framework Ga species likely contributes to the reduced BET surface area of Ga-zeolite catalysts compared to their Al-zeolite counterparts (Table 1); however, the percentage of Lewis acids among Ga- and Al-zeolites was generally the same (with the exception of Ga-MCM-22 that has much higher percentage of Lewis acid sites compared to Al-MCM-22).

As previously discussed, it has been established that the acid strength of Ga-zeolites is less than that of Al-zeolites (Meeprasert, J.; Jungsuttiwong, S.; Namuangruk, S., Location and acidity of Brønsted acid sites in isomorphously substituted LTL zeolite: A periodic density functional study. Microporous Mesoporous Mater. 2013, 175, 99-106). This is reflected in the NH₃-TPD data where the peaks for Ga-zeolites are shifted to much lower temperatures compared to those of their Al counterparts. The total acid site density calculated from NH₃-TPD (Table 1) also revealed significant differences between each Ga- and Al-zeolite isostructure due to the varying degree of the Si/M ratio used, with the exception of the MCM-22 materials where there was little to no change in total acid side density (e.g., 491 vs. 485 umol/g for Ga-MCM-22 and Al-MCM-22, respectively) suggesting that both gallium and aluminum occupy a similar number of acid sites at the same Si/M ratio. Moreover, there was only a marginal difference in the relative percentage of Lewis acids between Ga- and Al-zeolites, with the lone exception being Ga-MCM-22 which had 54% Lewis acidity compared to 15% for Al-MCM-22. For all samples, pyridine FTIR was used in accordance with previous studies in the literature where Brønsted and Lewis acid sites on solid acid catalysts are associated with peaks at 1540 cm⁻¹ and 1450 cm⁻¹, respectively (Emeis, C., Determination of integrated molar extinction coefficients for infrared absorption bands of pyridine adsorbed on solid acid catalysts. J. Catal. 1993, 147 (2), 347-354).

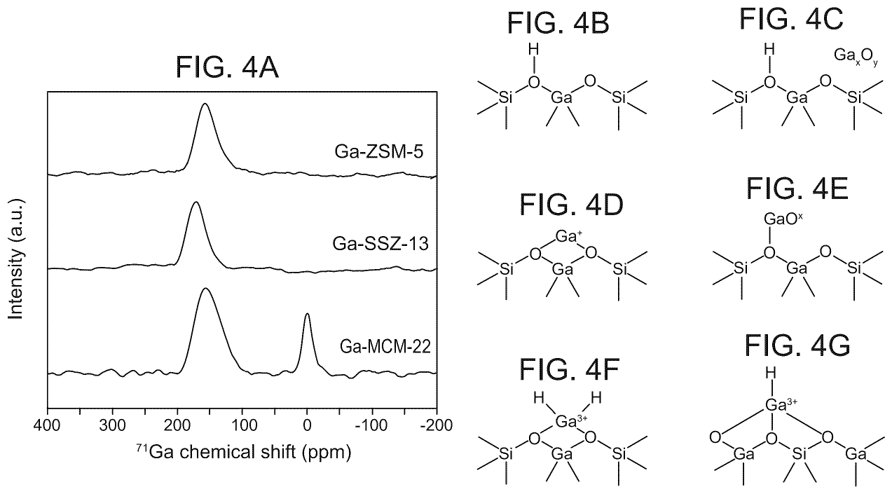

XPS analysis of Ga speciation is biased to the external surfaces of zeolite catalysts. In order to measure corresponding bulk properties, we used solid state magic angle spinning (MAS) ⁷¹Ga NMR for each Ga-zeolite (FIG. 4A). The ⁷¹Ga NMR spectra distinguish sites with either tetrahedral or non-tetrahedral coordination at peaks around 150 and 7 ppm, respectively. In a previous study (Zhou, Y., Thirumalai, H.; Smith, S. K.; Whitmire, K. H.; Liu, J., Frenkel, A. I.; Grabow, L. C.; Rimer, J. D., Ethylene dehydroaromatization over Ga-ZSM-5 catalysts: nature and role of gallium speciation. Angew. Chem. Int. Ed. 2020, 59 (44), 19592-19601), we showed that Ga-ZSM-5 contained only tetrahedrally-coordinated sites that corresponded to either framework (FIG. 4B) or extra-framework (FIG. 4F and FIG. 4G) species. The latter are consistent with Ga-zeolites prepared by post-synthesis processes. Evidence of extra-framework Ga sites neighboring multiple different framework sites (FIG. 4D-FIG. 4G) was not previously observed in the one-pot synthesis of Ga-ZSM-5, but extra-framework Ga³⁺ species have been reported in the literature (Phadke, N. M.; Mansoor, E.; Bondil, M., Head-Gordon, M.; Bell, A. T., Mechanism and kinetics of propane dehydrogenation and cracking over Ga/H-MFI prepared via vapor-phase exchange of H-MFI with GaCl3. J. Am. Chem. Soc. 2018, 141 (4), 1614-1627). For the Ga-zeolites prepared in the work disclosed herein, we observed only tetrahedral coordination for Ga-ZSM-5 and Ga-SSZ-13 (FIG. 4A). The percentage of framework and extra-framework species is not easily extracted from NMR data (Dai, W., Yang, L.; Wang, C. Wang, X.; Wu, G.; Guan, N.; Obenaus, U.; Hunger, M.; Li, L., Effect of n-butanol cofeeding on the methanol to aromatics conversion over Ga-modified nano H-ZSM-5 and its mechanistic interpretation. ACS Catal. 2018, 8 (2), 1352-1362; Arnold, A.; Steuernagel, S.; Hunger, M.; Weitkamp, J., Insight into the dry-gel synthesis of gallium-rich zeolite [Ga] Beta. Microporous Mesoporous Mater. 2003, 62 (1-2), 97-106), thus we were unable to differentiate the population of Ga species. For the Ga-MCM-22 sample, we observed non-tetrahedral extra-framework gallium species (FIG. 4A, FIG. 4C, and FIG. 4E) that are indicated by the 7 ppm peak. This is consistent with the small Bragg peaks of gallium oxide observed in the PXRD pattern (FIG. 11), the relatively high percentage of Lewis acidity (Table 1), and the highest fraction of extra-framework species detected in XPS data (FIG. 3A) among the three Ga-zeolite samples.

### Catalytic Performance: Methanol to Hydrocarbons

In the conversion of methanol to hydrocarbons (MTH), there has been extensive research on the key dehydration intermediate, dimethyl ether (DME), responsible for driving the first C—C bond formation and dictating hydrocarbon pool mechanistic pathways (Catizzone, E.; Aloise, A.; Migliori, M.; Giordano, G., Dimethyl ether synthesis via methanol dehydration: Effect of zeolite structure. Appl. Catal. A-Gen 2015, 502, 215-220; Fu, Y.; Hong, T.; Chen, J.; Auroux, A.; Shen, J., Surface acidity and the dehydration of methanol to dimethyl ether. Thermochim. Acta 2005, 434 (1-2), 22-26; Vishwanathan, V.; Jun, K.-W., Kim, J.-W.; Roh, H.-S., Vapour phase dehydration of crude methanol to dimethyl ether over Na-modified H-ZSM-5 catalysts. Appl. Catal. A-Gen 2004, 276 (1-2), 251-255). For this reason, prior literature reports investigating a combination of methanol (MeOH) and DME feeds (e.g., MeOH, DME, and DME/water) for MTH have shown that DME is significantly more reactive than methanol, enhances catalyst lifetime, and favors the olefin cycle pathway (Martinez-Espin, J. S.; Morten, M.; Janssens, T. V.; Svelle, S.; Beato, P.; Olsbye, U., New insights into catalyst deactivation and product distribution of zeolites in the methanol-to-hydrocarbons (MTH) reaction with methanol and dimethyl ether feeds. Catal. Sci. Technol. 2017, 7 (13), 2700-2716). Additionally, MTH catalyst deactivation has been linked to the formation of formaldehyde, known to be a coke precursor and a byproduct of methanol oxidation, which is bypassed when using DME as the feed. One limitation for the direct use of DME as the reagent is its high cost relative to that of methanol. In the work described herein, we explored the use of Ga-zeolites as selective dehydration catalysts for conversion of methanol to DME at conditions comparable to the MTH reaction (i.e., higher temperature than those required for conventional methanol dehydration catalysts). The use of Ga-zeolites capitalized on differences in Brønsted acidity wherein the substitution of Al with Ga results in weaker acid sites for methanol dehydration,

2MeOH↔DME+H₂O (1)

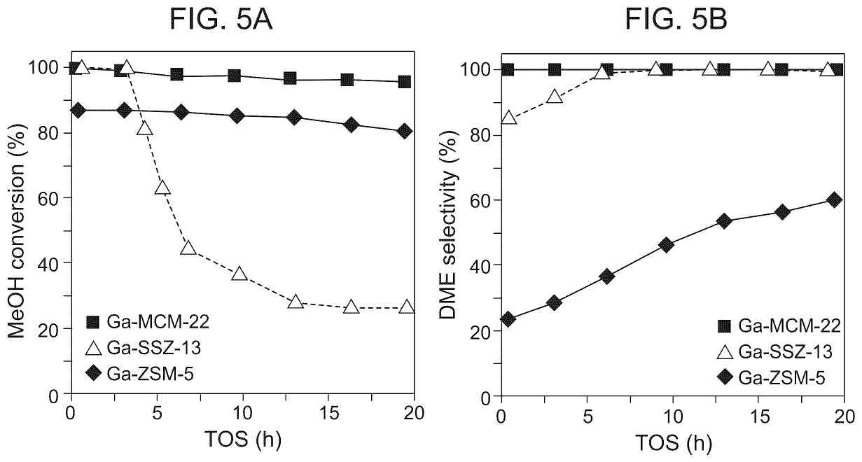

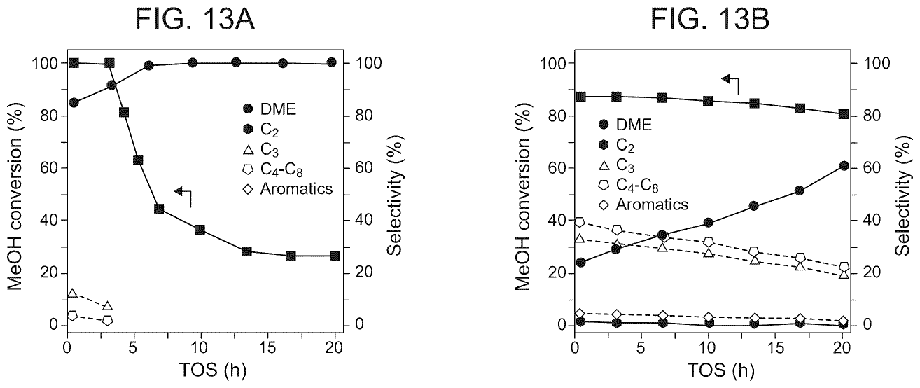

As shown in FIG. 5A-FIG. 5B, Ga-MCM-22 achieved approximately complete methanol conversion and DME selectivity at 350° C., which is within the upper range of temperatures employed in conventional methanol dehydration and falls within a typical range used for the MTH reaction. We only observed slight Ga-MCM-22 catalyst deactivation within 20 h of reaction time (FIG. 5A) where the methanol conversion decreased to 96% while DME selectivity remained fixed at 100% (FIG. 5B). The Ga-SSZ-13 catalyst had an initial conversion of 100% but deactivated upon exposure to methanol, reaching a steady state conversion of 28% within 15 h of reaction time. The DME selectivity over the Ga-SSZ-13 catalyst was initially lower (86%) due to the formation of C₃ and C₄ products (FIG. 13A-FIG. 13B), which putatively induced deactivation in the small-pore zeolite. As the catalyst deactivated, we observed a progressive increase in DME selectivity which reached 100% within 5 h reaction time (i.e., shortly after the catalyst begins to deactivate). The Ga-ZSM-5 catalyst exhibited lower activity than Ga-MCM-22, but similar to the latter there was only minimal deactivation over the total time on stream (FIG. 5A). There was, however, a noticeable difference in performance regarding DME selectivity (FIG. 5B), which monotonically increased with reaction time (from 24% to 60%) owing to the production of side products (primarily C₃ and C₄ species, FIG. 13A-FIG. 13B).

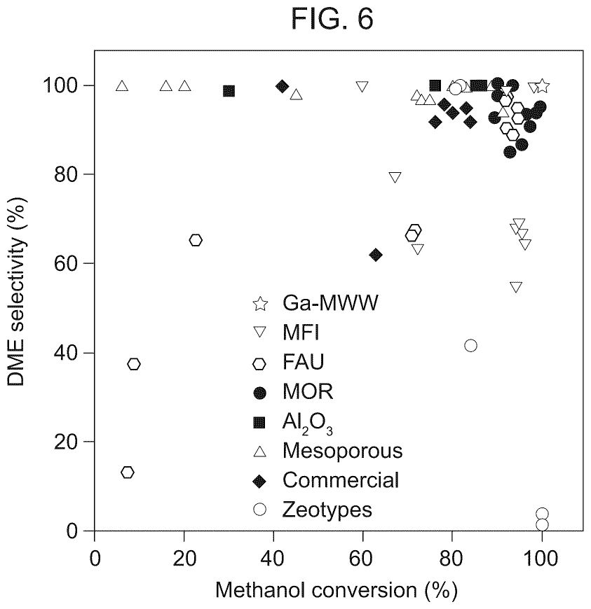

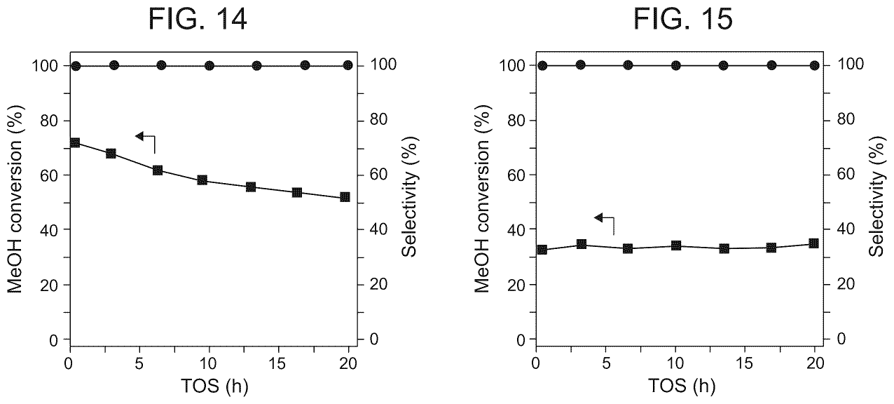

Surprisingly, we found that gallosilicate zeolites—notably Ga-MCM-22—were exceptional catalysts for methanol dehydration given the high methanol conversion and DME selectivity relative to conventional catalysts over a broad range of reaction temperatures, spanning from 250° C. (FIG. 14) to 350° C. (FIG. 6). In FIG. 6 we compared the methanol conversion and DME selectivity of Ga-MCM-22 to catalysts reported in literature (see Table 2 for details of each catalyst and corresponding reaction conditions).

TABLE 2
Reaction conditions of methanol dehydration catalysts extracted from
prior literature reports. Catalysts are grouped according to the catalyst type provided in catalyst
type FIG. 6.

| | | | | MeOH | DME | Catalyst | |
|---|---|---|---|---|---|---|---|
| | | Feed | | conv. | select. | Type | |
| Catalyst | T (° C.) | composition | Feed rate | (%) | (%) | (FIG. 6) | Ref. |
| γ-Al₂O₃ | 275 | MeOH | 5.6 g h | 30 | 98.7 | Al₂O₃ | 1 |
| | | | mol⁻¹ | | | | |
| γ-Al₂O₃ | 290 | MeOH | (LHSV) | 86.1 | 100 | Al₂O₃ | 1 |
| | | | 0.9 h⁻¹ | | | | |
| γ-Al₂O₃ | 300 | 11% MeOH | (WHSV) | 76.2 | 100 | Al₂O₃ | 1 |
| | | in N₂ | 1.0 h⁻¹ | | | | |
| 5% Ti(SO₄)₂/γ- | 240 | 21% MeOH | (GHSV) | 85 | 100 | Al₂O₃ | 1 |
| Al₂O₃ | | in N₂ | 3.4 L g⁻¹ | | | | |
| | | | h⁻¹ | | | | |
| Boria-γ-Al₂O₃ | 350 | 0.1% MeOH | (WHSV) | 85 | 100 | Al₂O₃ | 1 |
| | | in N₂ | 4.0 h⁻¹ | | | | |
| Fluorinated-γ- | 400 | MeOH | 4.98*10⁻² | 82 | 100 | Al₂O₃ | 1 |
| Al₂O₃ | | | mol h⁻¹ | | | | |
| Chlorinated-γ- | 400 | MeOH | 4.98*10⁻² | 90 | 100 | Al₂O₃ | 1 |
| Al₂O₃ | | | mol h⁻¹ | | | | |
| Na-modified H- | 270 | MeOH | (LHSV) | 83 | 100 | MFI | 1 |
| ZSM-5 | | (crude) in N₂ | 10 h⁻¹ | | | | |
| NaHZSM-5/γ- | 250 | MeOH | (LHSV) | 82 | 100 | MFI | 1 |
| Al₂O₃ | | (crude) in N₂ | 10 h⁻¹ | | | | |
| NaH-ZSM-5 | 250 | MeOH | (WHSV) | 60 | 100 | MFI | 1 |
| | | | 4.0 h⁻¹ | | | | |
| Na-modified H- | 300 | MeOH | (LHSV) | 98 | 100 | MFI | 1 |
| ZSM-5 | | | 3.8 h⁻¹ | | | | |
| NaH-ZSM-5 | 260 | MeOH | (ST) 5.6 g | 90 | 99 | MFI | 1 |
| | | | h mol⁻¹ | | | | |
| NaOH-treated | 260 | MeOH | (ST) 5.6 g | 92 | 99 | MFI | 1 |
| NH₄-ZSM-5 | | | h mol⁻¹ | | | | |
| HFeZSM-5 | 260 | 66.6 H₂ | (GHSV) | 94.1 | 54.9 | MFI | 1 |
| | | 33.3% CO | 1500 cm³ | | | | |
| | | | g⁻¹ h⁻¹ | | | | |
| HFeAlZSM-5 | 260 | 66.6 H₂ | (GHSV) | 95.5 | 67.1 | MFI | 1 |
| | | 33.3% CO | 1500 cm³ | | | | |
| | | | g⁻¹ h⁻¹ | | | | |
| H-ZSM- | 220 | MeOH | 0.1 mL | 90 | 100 | MFI | 1 |
| 5/MCM-41 | | | min⁻¹ | | | | |
| MgO-modified | 260 | 66% H₂, 30% | 1500 mL | 96.3 | 64.5 | MFI | 1 |
| H-ZSM-5 | | CO, 4% CO₂ | g⁻¹ h⁻¹ | | | | |
| Sb₂O₃-modified | 260 | 61.4% H₂ | 1500 mL | 95 | 69 | MFI | 1 |
| H-ZSM-5 | | 28.5% CO, | g⁻¹ h⁻¹ | | | | |
| | | 2.8% CO₂, | | | | | |
| | | 7.3% N₂ | | | | | |
| Silylated H- | 260 | 66.6% H₂, | (WHSV) | 72 | 63.5 | MFI | 1 |
| ZSM-5 | | 33.3% CO | 1.7 h⁻¹ | | | | |
| H-ZSM-5/H-Y | 250 | 60.8% H₂, | (SV) | 94.2 | 67.9 | MFI | 1 |
| | | 27.2% CO, | 1500 h⁻¹ | | | | |
| | | 4.8% CO₂, | | | | | |
| | | 3.2% Ar | | | | | |
| Fe-modified H- | 245 | 60% H₂, | (SV) | 22.6 | 65.2 | FAU | 1 |
| Y | | 40% CO | 1500 h⁻¹ | | | | |
| Co-modified H- | 245 | 60% H₂, | (SV) | 8.7 | 37.5 | FAU | 1 |
| Y | | 40% CO | 1500 h⁻¹ | | | | |
| Ni-modified H- | 245 | 60% H₂, 40% | (SV) | 7.5 | 13.3 | FAU | 1 |
| Y | | CO | 1500 h⁻¹ | | | | |
| Cr-modified H- | 245 | 60% H₂, 40% | (SV) | 70.9 | 66.7 | FAU | 1 |
| Y | | CO | 1500 h⁻¹ | | | | |
| Zr-modified H- | 245 | 60% H₂, 40% | (SV) | 71.4 | 67.5 | FAU | 1 |
| Y | | CO | 1500 h⁻¹ | | | | |
| La-modified H- | 245 | 20% MeOH | 50 mL | 92.2 | 97.4 | FAU | 1 |
| Y | | in Ar | min⁻¹ | | | | |
| Ce-modified H- | 245 | 20% MeOH | 50 mL | 94.5 | 94.7 | FAU | 1 |
| Y | | in Ar | min⁻¹ | | | | |
| Pr-modified H- | 245 | 20% MeOH | 50 mL | 92 | 96.7 | FAU | 1 |
| Y | | in Ar | min⁻¹ | | | | |
| Nd-modified H- | 245 | 20% MeOH | 50 mL | 94.6 | 92.7 | FAU | 1 |
| Y | | in Ar | min⁻¹ | | | | |
| Sm-modified H- | 245 | 20% MeOH | 50 mL | 93.4 | 89 | FAU | 1 |
| Y | | in Ar | min⁻¹ | | | | |
| Eu-modified H- | 245 | 20% MeOH | 50 mL | 92 | 90.5 | FAU | 1 |
| Y | | in Ar | min⁻¹ | | | | |
| Cu-H-MOR | 250 | MeOH in | 0.926 | 95.1 | 87.1 | MOR | 1 |
| | | isooctane | mol L⁻¹ | | | | |
| Mg-H-MOR | 250 | MeOH in | 0.926 | 92.9 | 85.2 | MOR | 1 |
| | | isooctane | mol L⁻¹ | | | | |
| Ni-H-MOR | 250 | MeOH in | 0.926 | 89.5 | 92.9 | MOR | 1 |
| | | isooctane | mol L⁻¹ | | | | |
| Zn-H-MOR | 250 | MeOH in | 0.926 | 97.4 | 90.8 | MOR | 1 |
| | | isooctane | mol L⁻¹ | | | | |
| Al-H-MOR | 250 | MeOH in | 0.926 | 99.4 | 95.2 | MOR | 1 |
| | | isooctane | mol L⁻¹ | | | | |
| Zr-H-MOR | 250 | MeOH in | 0.926 | 98.8 | 94.1 | MOR | 1 |
| | | isooctane | mol L⁻¹ | | | | |
| Na-H-MOR | 250 | MeOH in | 0.926 | 96.2 | 93.5 | MOR | 1 |
| | | isooctane | mol L⁻¹ | | | | |
| Fluorinated H- | 300 | MeOH in Ar | 4.98*10⁻² | 90 | 98 | MOR | 1 |
| MOR | | | mol h⁻¹ | | | | |
| Chlorinated H- | 300 | MeOH in Ar | 4.98*10⁻² | 93 | 100 | MOR | 1 |
| MOR | | | mol h⁻¹ | | | | |
| H-MOR/H-B | 200 | MeOH in Ar | (WHSV) | 90 | 100 | MOR | 1 |
| | | | 2.26 g h⁻¹ | | | | |
| MCM-41 | 400 | 97.48 mmHg | 14 L g⁻¹ h⁻¹ | 6 | 100 | Mesoporous | 1 |
| | | MeOH in N₂ | | | | | |
| Al-MCM-41 | 400 | 97.48 mmHg | 14 L⁻¹ g⁻¹ h⁻¹ | 45 | 98 | Mesoporous | 1 |
| | | MeOH in N₂ | | | | | |
| Al-MCM-41 | 400 | 97.48 mmHg | 14 L g⁻¹ h⁻¹ | 72 | 98 | Mesoporous | 1 |
| | | MeOH in N₂ | | | | | |
| Al-MCM-41 | 400 | 97.48 mmHg | 14 L g⁻¹ h⁻¹ | 73 | 97 | Mesoporous | 1 |
| | | MeOH in N₂ | | | | | |
| Al-HMS-5 | 300 | 11% MeOH | (WHSV) | 91.2 | 94.1 | Mesoporous | 1 |
| | | in N₂ | 1.0 h⁻¹ | | | | |
| Al-HMS-10 | 300 | 11% MeOH | (WHSV) | 89 | 100 | Mesoporous | 1 |
| | | in N₂ | 1.0 h⁻¹ | | | | |
| Al-HMS-20 | 325 | 11% MeOH | (WHSV) | 20 | 100 | Mesoporous | 1 |
| | | in N₂ | 1.0 h⁻¹ | | | | |
| Al-HMS-35 | 375 | 11% MeOH | (WHSV) | 16 | 100 | Mesoporous | 1 |
| | | in N₂ | 1.0 h⁻¹ | | | | |
| Al-SBA-15 | 350 | 50% MeOH | 13.3 L | 80 | 100 | Mesoporous | 1 |
| | | in N₂ | g⁻¹ h⁻¹ | | | | |
| SBA-15-SO₃H- | 300 | 4% MeOH in | 20 ml | 83 | 100 | Mesoporous | 1 |
| Al | | N₂ | min⁻¹ | | | | |
| MCF-SO₃H-Al | 300 | 4% MeOH in | 20 ml | 82 | 100 | Mesoporous | 1 |
| | | N₂ | min⁻¹ | | | | |
| MSU-S | 380 | MeOH | (WHSV) | 75 | 97 | Mesoporous | 1 |
| | | | 5.0 h⁻¹ | | | | |
| Al-MFI | 240 | MeOH | (WHSV) | 80 | 94 | Commercial | 2 |
| | | | 2.0 h⁻¹ | | | | |
| Al-FER | 240 | MeOH | (WHSV) | 83 | 95 | Commercial | 2 |
| | | | 2.0 h⁻¹ | | | | |
| Al-MOR | 240 | MeOH | (WHSV) | 84 | 92 | Commercial | 2 |
| | | | 2.0 h⁻¹ | | | | |
| Al-BEA | 240 | MeOH | (WHSV) | 76 | 92 | Commercial | 2 |
| | | | 2.0 h⁻¹ | | | | |
| Al-BEA | 300 | MeOH | (WHSV) | 63 | 62 | Commercial | 2 |
| | | | 2.0 h⁻¹ | | | | |
| Al-FER | 240 | MeOH | (WHSV) | 42 | 100 | Commercial | 2 |
| | | | 2.0 h⁻¹ | | | | |
| Al-FER | 300 | MeOH | (WHSV) | 78 | 96 | Commercial | 2 |
| | | | 2.0 h⁻¹ | | | | |
| SAPO-5 | 350 | MeOH | (WHSV) | 100 | 1.1 | Zeotypes | 1 |
| | | | 1.0 h⁻¹ | | | | |
| SAPO-11 | 350 | MeOH | (WHSV) | 84.2 | 41.7 | Zeotypes | 1 |
| | | | 1.0 h⁻¹ | | | | |
| SAPO-41 | 350 | MeOH | (WHSV) | 100 | 3.7 | Zeotypes | 1 |
| | | | 1.0 h⁻¹ | | | | |
| AlPO-5 | 350 | MeOH | (WHSV) | 81 | 99.6 | Zeotypes | 1 |
| | | | 1.0 h⁻¹ | | | | |
| AlPO-11 | 350 | MeOH | (WHSV) | 81.4 | 99.9 | Zeotypes | 1 |
| | | | 1.0 h⁻¹ | | | | |
| AlPO-41 | 350 | MeOH | (WHSV) | 82.4 | 99.9 | Zeotypes | 1 |
| | | | 1.0 h⁻¹ | | | | |

Ref. 1: Bateni, H.; Able, C., Catal. Ind. 2019, 11, 7-33. Ref. 2: Catizzone, E.; Aloise, A.; Migliori, M.; Giordano, G., Appl. Catal. A-Gen 2015, 502, 215-220.

The majority of catalysts operate in the range of 200-290° C., while approximately one-third of the catalysts have been tested in the range of 300-400° C. The latter are largely non-zeolitic materials, whereas the majority of reactions involving zeolite catalysts tend to operate below 300° C., which includes commercial Al-zeolite catalysts MFI, FER, MOR, and *BEA (Catizzone, E.; Aloise, A.; Migliori, M.; Giordano, G., Dimethyl ether synthesis via methanol dehydration: Effect of zeolite structure. Appl. Catal. A-Gen 2015, 502, 215-220). When conventional Al-zeolite catalysts are used at higher temperatures, their strong Brønsted acidity cannot prevent subsequent reactions of DME by the hydrocarbon pool mechanism. In order to achieve higher methanol conversion without sacrificing DME selectivity, studies have investigated zeolites (e.g., MFI, FAU, and MOR) where their acid site density is reduced by methods that include functionalization (fluorination, chlorination, silylation), exchanged oxides (MgO, Sb₂O₃), and exchanged cations/metals (Na, Fe, Co, Ni, Cr, Zr, La, etc.) (Fei, J.; Hou, Z.; Zhu, B.; Lou, H.; Zheng, X., Synthesis of dimethyl ether (DME) on modified HY zeolite and modified HY zeolite-supported Cu—Mn—Zn catalysts. Appl. Catal. A-Gen 2006, 304, 49-54; Xia, J.; Mao, D.; Zhang, B.; Chen, Q.; Zhang, Y., Tang, Y., Catalytic properties of fluorinated alumina for the production of dimethyl ether. Catal. Commun. 2006, 7 (6), 362-366; Ryndin, Y. A.; Hicks, R.; Bell, A.; Yermakov, Y. I., Effects of metal-support interactions on the synthesis of methanol over palladium. J. Catal. 1981, 70 (2), 287-297). Similar approaches have been used for non-zeolitic materials, such as γ-Al₂O₃ with functionalization (fluorination, chlorination) and doping with oxides (e.g., SiO₂, Ti(SO₄)₂, B₂O₃, and ZrO₂). Others include various mesoporous materials (MCM-41, SBA-15, HMS, MCF, and MSU) with and without dopants (e.g., Al) (Sabour, B.; Peyrovi, M. H.; Hamoule, T.; Rashidzadeh, M., Catalytic dehydration of methanol to dimethyl ether (DME) over Al-HMS catalysts. J. Ind. Eng. Chem. 2014, 20 (1), 222-227; Azimov, F.; Markova, I; Stefanova, V.; Sharipov, K., Synthesis and characterization of SBA-15 and Ti-SBA-15 nanoporous materials for DME catalysts. J. Chem. Technol. Metall. 2012, 47 (3), 333-340). Unexpectedly, the performance of Ga-MCM-22 (gold star in FIG. 6) is superior to other catalysts, noting that conventional materials are typically evaluated at much longer methanol contact times (e.g., WHSV=1 to 4 h⁻¹) compared to those in this study (WHSV=8 h⁻¹).

A distinguishing aspect of Ga-zeolites compared to others in FIG. 6 is the methodology by which their acidity is tuned. Notably, here we replaced framework Al with a less acidic heteroatom Ga. This is counter to methods previously reported in the literature where acid site density is reduced by incorporation of extra-framework species to replace protons (i.e., Brønsted acid sites). If we compare the performance of Ga-MCM-22 to SAPO and AlPO zeotypes at the same reaction temperature (350° C., FIG. 6), it is reported in the literature that AlPOs (e.g., AIPO-5, AIPO-11, and AIPO-41) can achieve approximately 100% DME selectivity but cannot match the methanol conversion of Ga-MCM-22 (Bateni, H.; Able, C., Development of heterogeneous catalysts for dehydration of methanol to dimethyl ether: A review. Catal. Ind. 2019, 11, 7-33). Similarly, as reported in the literature SAPOs (SAPO-5, SAPO-11, SAPO-41) can attain 100% DME selectivity as methanol dehydration catalysts at lower temperatures but become less effective at 350° C. (e.g., with SAPO-11 exhibiting the best performance among all SAPOs with 84% methanol conversion and 42% DME selectivity) (Bateni, H.; Able, C., Development of heterogeneous catalysts for dehydration of methanol to dimethyl ether: A review. Catal. Ind. 2019, 11, 7-33).

Prior studies reported in the literature have shown that DME selectivity is directly correlated with less acidic sites and inversely correlated with strong acid sites (Fu, Y.; Hong, T.; Chen, J.; Auroux, A.; Shen, J., Surface acidity and the dehydration of methanol to dimethyl ether. Thermochim. Acta 2005, 434 (1-2), 22-26; Carr, R. T.; Neurock, M.; Iglesia, E., Catalytic consequences of acid strength in the conversion of methanol to dimethyl ether. J. Catal. 2011, 278 (1), 78-93). The relative roles of Brønsted and Lewis acid sites in methanol dehydration has been considered where it is generally believed that Brønsted acid sites are more responsible for DME formation (Carr, R. T.; Neurock, M.; Iglesia, E., Catalytic consequences of acid strength in the conversion of methanol to dimethyl ether. J. Catal. 2011, 278 (1), 78-93; Bakhtyari, A.; Rahimpour, M. R., Methanol to dimethyl ether. In Methanol, Elsevier: 2018; pp 281-311). Functionalization and exchange of Al-zeolite catalysts reduces the number of strong Brønsted acid sites, which achieves higher DME selectivity at the expense of reduced activity, thus requiring longer contact times to achieve higher methanol conversion. The reduced Brønsted acidity of Ga-zeolites allows for high conversion and selectivity at much shorter contact times. The three Ga-zeolites examined in the work described herein also contain a large percentage of Lewis acid sites relative to their Al-zeolite counterparts. It is not well understood what role these species contribute to methanol dehydration; however, it is interesting to note that there are clear differences between the performance of the three Ga-zeolites that suggest, without being bound by theory, that there are factors beyond replacing Al with Ga that contribute to the activity of the catalyst. Ga-MCM-22 is a standout among the zeolite frameworks selected for the work described herein. One notable difference of Ga-MCM-22 is the presence of a small quantity of gallium oxide Davis et al. (Davis, B. H.; Cook, S.; Naylor, R. W., Catalytic conversion of alcohols. 8. Gallium oxide as a dehydration catalyst. J. Org. Chem. 1979, 44 (13), 2142-2145) reported the activity of gallium oxide for the dehydration of numerous alcohols (e.g., propanol and butanol) at low temperature (≤200° C.) leads to moderate conversions (<50%). Izzo et al. (Izzo, L.; Tabanelli, T.; Cavani, F.; Vásquez, P. B.; Lucarelli, C.; Mella, M., The competition between dehydrogenation and dehydration reactions for primary and secondary alcohols over gallia: unravelling the effects of molecular and electronic structure via a two-pronged theoretical/experimental approach. Catal. Sci. Technol. 2020, 10 (10), 3433-3449) explored the competing processes of dehydration and dehydrogenation for methanol over a gallium oxide catalyst at higher temperature (400° C.) and observed low DME selectivity (ca. 35%) with a range of byproducts (e.g., CO₂, CO, CH₄, CH₂O, H₂) that are not observed in our reactions with Ga-zeolites. This suggests gallium oxide may exhibit a small contribution to total methanol turnovers but cannot account for the exceptional selectivity of Ga-MCM-22. To confirm this hypothesis, without being bound by theory, we tested commercially available Ga₂O₃ for methanol dehydration at identical reaction conditions to that of the Ga-zeolites and found that the catalyst exhibits moderate conversion (˜35%) with 100% DME selectivity (FIG. 15) over 20 h time on stream. Without being bound by theory, these findings suggest that extra-framework Ga₂O₃ in Ga-MCM-22 may contribute to the overall catalytic performance, but additional Ga sites are needed to achieve both 100% conversion and 100% DME selectivity. It remains to be determined what factors differentiate the unique performance of Ga-MCM-22 over those of Ga-SSZ-13 and Ga-ZSM-5. Without being bound by theory, it is likely that there are differences in Ga speciation (and hence acid strength) that cannot be easily discerned by common analytical techniques; or perhaps there are effects associated with crystal structure (i.e., confinement) and/or the distribution of Ga species (i.e., acid siting).

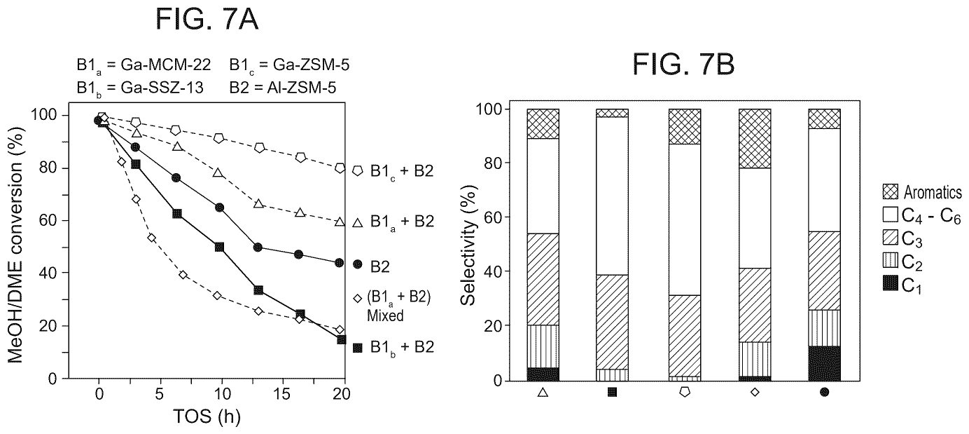

The relatively weak acid strength of Ga-zeolites enables existing fixed bed reactors utilizing Al-zeolites at high temperatures to be easily configured as an integrated dual bed system where the Ga-zeolite (placed upstream as bed B1) functions as a pre-treatment catalyst to convert a cheaper feed into one that is a more efficient reactant for the Al-zeolite catalyst (placed downstream as bed B2). This general reactor configuration is illustrated in FIG. 1. Here we compared a series of tandem catalysts using three different Ga-zeolites upstream for methanol dehydration and Al-ZSM-5 downstream for the conversion of DME to hydrocarbons (FIG. 7A). Plots of methanol/DME conversion versus time on stream revealed the following trends in the deactivation rates of tandem catalysts: Ga-SSZ-13>Ga-MCM-22>Ga-ZSM-5. The Ga-MCM-22(B1)-Al-ZSM-5(B2) dual bed exhibits a longer catalyst lifetime than the single bed of Al-ZSM-5 (control) as a result of a 100% DME selectivity upstream from the methanol dehydration catalyst. The Ga-ZSM-5(B1)-Al-ZSM-5(B2) dual bed resulted in the longest catalyst lifetime owing to a reduced DME and increased light olefins content of the feed sent to the downstream catalyst. This is reflected in the product selectivity (FIG. 7B) with a higher percentage of C₃ and C₄ species (i.e., the byproducts of methanol dehydration upstream, FIG. 13A-FIG. 13B); and it is also consistent with the overall yield of propylene, which was highest for the Ga-ZSM-5(B1)-Al-ZSM-5(B2) dual bed configuration. We also assessed a single bed containing a physical mixture of Ga-MCM-22 and Al-ZSM-5 and observed a dramatic reduction in catalyst lifetime compared to both the control and dual bed configurations.

Previous literature studies have reported increased catalyst lifetime when DME is introduced as the reactant instead of methanol (Martinez-Espin, J. S.; Morten, M.; Janssens, T. V. W.; Svelle, S.; Beato, P.; Olsbye, U.; New insights into catalyst deactivation and product distribution of zeolites in the methanol-to-hydrocarbons (MTH) reaction with methanol and dimethyl ether feeds. Catal. Sci. Technol. 2017, 7, 2700-2716), which can explain the improved performance of dual bed configurations; however, a switch from the best system when Ga-MCM-22 is placed upstream of Al-ZSM-S to the worst system when the two catalysts are added as a physical mixture is unexpected. Although the exact mechanism(s) for the trends observed in FIG. 7A is elusive, evidence can be gleaned from differences in product distributions at early time on stream (FIG. 7B). The physical mixture produces the highest aromatics selectivity, which accelerates the rate of coking (Hwang, A.; Bhan, A., Deactivation of zeolites and zeotypes in methanol-to-hydrocarbons catalysis: Mechanisms and circumvention. Acc. Chem. Res. 2019, 52 (9), 2647-2656; Wang, C.; Hu, M.; Chu, Y.; Zhou, X.; Wang, Q.; Qi, G., Li, S.; Xu, J.; Deng, F., π-Interactions between Cyclic Carbocations and Aromatics Cause Zeolite Deactivation in Methanol-to-Hydrocarbon Conversion. Angew. Chem. Int. Ed. 2020, 59 (18), 7198-7202). Moreover, our previous study of Ga/Al-ZSM-5 zeolites (Zhou, Y.; Thirumalai, H., Smith, S. K.; Whitmire, K. H.; Liu, J.; Frenkel, A. I.; Grabow, L. C.; Rimer, J. D., Ethylene dehydroaromatization over Ga-ZSM-5 catalysts: nature and role of gallium speciation. Angew. Chem. Int. Ed. 2020, 59 (44), 19592-19601) demonstrated that Ga species promote aromatization, which can explain why the physical mixture of both Ga- and Al-zeolites increases the rate of catalyst deactivation relative to that of the dual bed configuration. When comparing the latter to the single bed of Al-ZSM-5, the effluent from the dual bed contains less methane, consistent with a reduced rate of catalyst deactivation. We also observed higher C₃ and C₂ selectivities for the Ga-MCM-22(B1)-Al-ZSM-5(B2) dual bed, which is consistent with the study by Martinez-Espin et al. (Martinez-Espin, J. S.; Morten, M.; Janssens, T. V.; Svelle, S.; Beato, P.; Olsbye, U., New insights into catalyst deactivation and product distribution of zeolites in the methanol-to-hydrocarbons (MTH) reaction with methanol and dimethyl ether feeds. Catal. Sci. Technol. 2017, 7 (13), 2700-2716) showing a higher selectivity to light olefins when methanol is replaced with DME as the reactant.

A comparison of selectivities (FIG. 7B) revealed the following trend in C₃/C₂ ratio (from lowest to highest): Ga-MCM-22/Al-ZSM-5 (mixed)<Al-ZSM-5<Ga-MCM-22(B1)-Al-ZSM-5(B2)<Ga-SSZ-13(B1)-Al-ZSM-5(B2)<Ga-ZSM-5(B1)-Al-ZSM-5(B2). The yield of propylene was highest for the Ga-MCM-22(B1)-Al-ZSM-5(B2) dual bed configuration. In terms of aromatics, the configurations that result in the largest rate of catalyst deactivation, Ga-SSZ-13(B1)-Al-ZSM-5(B2) and Ga-MCM-22/Al-ZSM-5 (mixed), had the lowest and highest aromatics selectivity, respectively. Without being bound by theory, we posit that the likely cause for a short lifetime in the former configuration is the low production of DME from the upstream Ga-SSZ-13 catalyst, and possibly the generation of pre-coking species (carried to the downstream catalyst) owing to confinement effects in the small pore CHA-type zeolite. For the physical mixture, there is a high percentage of methanol throughout the catalyst bed, which likely contributes to a relatively faster rate of catalyst deactivation in addition to gallium's promotion of aromatic species (i.e., possible coke precursors).

A consequence of the methanol dehydration reaction in the upstream bed is the formation of water, which is known to decrease the rate of MTH reactions and subsequently form coke deposits leading to increased downstream catalyst deactivation. The presence of water in feeds to the Al-ZSM-5 catalyst (bed B2) would be expected to reduce the efficiency of the tandem reactor design over what could be achieved by using a pure (anhydrous) DME feed; however, the improved performance of the dual-bed Ga-MCM-22/Al-ZSM-S and Ga-ZSM-5/AI-ZSM-5 configurations over that of a single Al-ZSM-5 bed (FIG. 7A) revealed that the positive effects of converting methanol to DME outweighs the negative impact of water generated from the upstream reaction. This is consistent with prior reports demonstrating the carbon conversion capacity is 8.3-times higher for a DME/water feed compared to a pure methanol feed (Perez-Uriarte, P.; Ateka, A.; Gamero, M.; Aguayo, A. T.; Bilbao, J., Effect of the Operating Conditions in the Transformation of DME to olefins over a HZSM-5 Zeolite Catalyst. Ind. Eng. Chem. Res. 2016, 55 (23), 6569-6578; Martinez-Espin, J. S.; Morten, M.; Janssens, T. V.; Svelle, S.; Beato, P.; Olsbye, U., New insights into catalyst deactivation and product distribution of zeolites in the methanol-to-hydrocarbons (MTH) reaction with methanol and dimethyl ether feeds. Catal. Sci. Technol. 2017, 7 (13), 2700-2716; Migliori, M., Catizzone, E.; Aloise, A.; Bonura, G.; Gómez-Hortigüela, L.; Frusteri, L.; Cannilla, C.; Frusteri, F.; Giordano, G., New insights about coke deposition in methanol-to-DME reaction over MOR-, MFI- and FER-type zeolites. J. Ind. Eng. Chem. 2018, 68, 196-208).

### Catalytic Performance: Ethanol to Propylene.

In our work described herein, we tested the broader applicability of Ga-zeolites in alcohol dehydration reactions by assessing ethanol (EtOH), which selectively produces ethylene,

EtOH↔C₂H₄+H₂O (2)

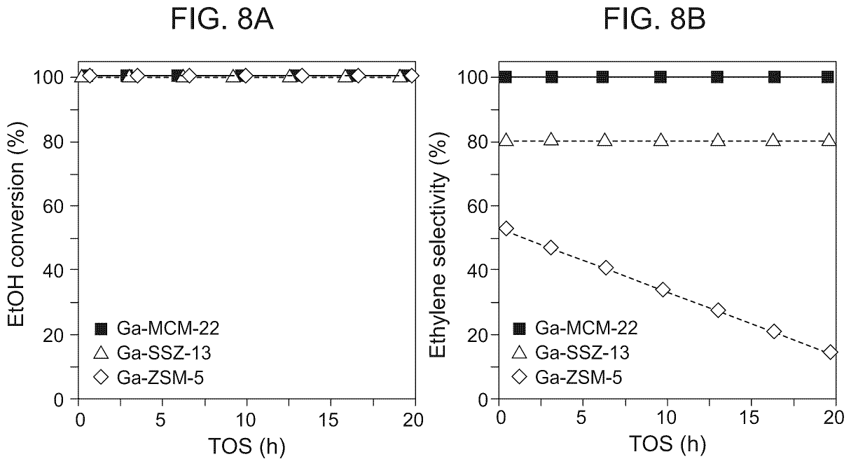

Most industrial processes use alumina-based catalysts (e.g., γ-Al₂O₃) for this reaction whereas zeolites are less studied; however, it has been shown that certain zeolites (e.g., ZSM-5, SAPO-34) exhibit higher activity than conventional ethanol dehydration catalysts (Zhang, M.; Yu, Y., Dehydration of ethanol to ethylene. Ind. Eng. Chem. Res. 2013, 52 (28), 9505-9514.; Zhou, X.; Wang, C.; Chu, Y., Xu, J.; Wang, Q.; Qi, G.; Zhao, X.; Feng, N.; Deng, F., Observation of an oxonium ion intermediate in ethanol dehydration to ethene on zeolite. Nat. Commun. 2019, 10(1), 1961; Phung, T. K.; Hernández, L. P.; Lagazzo, A.; Busca, G., Dehydration of ethanol over zeolites, silica alumina and alumina: Lewis acidity, Brønsted acidity and confinement effects. Appl. Catal. A-Gen 2015, 493, 77-89). Moreover, zeolites can operate at optimal reaction conditions, such as lower temperatures. One of the challenges facing zeolites is catalyst stability owing to their propensity to coke. Without being bound by theory, we hypothesize that Ga-zeolites with reduced acid site strength could increase catalyst lifetime beyond what has been observed for Al-zeolites, while also enabling operation at higher temperatures without sacrificing ethylene selectivity, similar to our findings for methanol dehydration. A typical range of reaction temperature for ethanol dehydration is 300-500° C. (Zhang, M.; Yu, Y., Dehydration of ethanol to ethylene. Ind. Eng. Chem. Res. 2013, 52 (28), 9505-9514) For the work described herein we selected the uppermost temperature with WHSV=19 h⁻¹, which is a significantly shorter contact time compared to conventional alumina-based catalysts that typically operate at WHSV=0.1-1.0 h⁻¹ (Zhang, M., Yu, Y., Dehydration of ethanol to ethylene. Ind. Eng. Chem. Res. 2013, 52 (28), 9505-9514) In the work described herein, we compared the performance of all three Ga-zeolites (Ga-MCM-22, Ga-ZSM-5, and Ga-SSZ-13) in terms of ethanol conversion and ethylene selectivity. The activity of all catalysts was comparable given the observation of 100% ethanol conversion (FIG. 8A) over a 20 h reaction time. Surprisingly, we saw notable differences in product selectivity (FIG. 8B) with Ga-MCM-22 exhibiting 100% ethylene selectivity. The Ga-SSZ-13 catalyst exhibited 80% ethylene selectivity, with propylene (15%) and C₄-(5%) as the undesired products. For Ga-ZSM-5 there was a progressive decrease in ethylene selectivity (from 52 to 15%) over the course of the reaction with a relatively constant propylene selectivity (25-30%), a small amount of ethane (<7%), and a progressive increase in C₄₌ selectivity (from 16 to 61%).

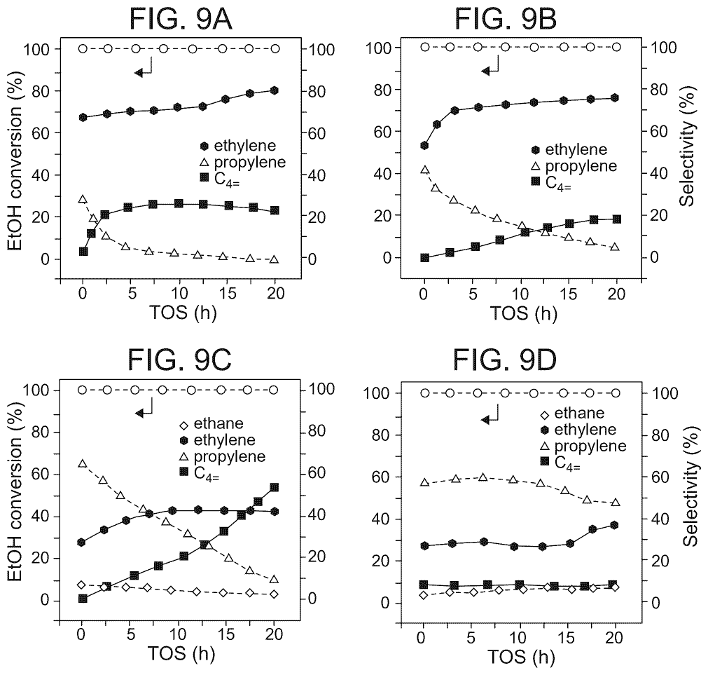

Similar to the approach used for the MTH reactor dual-bed, herein we used a similar configuration for the conversion of ethanol to propylene. In this design the upstream catalyst (Ga-zeolite) converts a cheap feedstock (ethanol) to a more value-added intermediate (ethylene) that without being bound by theory, we posit is more reactive for conversion downstream (Al-zeolite) to the desired product. Prior studies have examined ethanol to propylene reactions using CHA-type zeolites (Dai, W.; Sun, X.; Tang, B.; Wu, G.; Li, L., Guan, N.; Hunger, M., Verifying the mechanism of the ethene-to-propene conversion on zeolite H-SSZ-13. J. Catal. 2014, 314, 10-20; Lehmann, T., Seidel-Morgenstern, A., Thermodynamic appraisal of the gas phase conversion of ethylene or ethanol to propylene. J. Chem. Eng. 2014, 242, 422-432). Lehmann and Seidel-Morgenstern (Lehmann, T.; Seidel-Morgenstern, A., Thermodynamic appraisal of the gas phase conversion of ethylene or ethanol to propylene. J. Chem. Eng. 2014, 242, 422-432) proposed a mechanism for this reaction where propylene is always generated directly from ethylene rather than ethanol, thus requiring an initial dehydration step. Hunger et al. (Dai, W.; Sun, X.; Tang, B.; Wu, G.; Li, L.; Guan, N.; Hunger, M., Verifying the mechanism of the ethene-to-propene conversion on zeolite H-SSZ-13. J. Catal. 2014, 314, 10-20) also reported CHA-type zeolite (Al-SSZ-13) to exhibit the highest adsorption capacity and best activity towards propylene in ethylene to propylene among several other Al-zeolites (e.g., MOR, *BEA, MFI). As a benchmark, we first assessed a single bed reactor containing Al-SSZ-13 catalyst (FIG. 9A) and observed a decreasing ethylene conversion from 40 to 20% over 20 h time on stream. As provided herein, we have opted to report this data with respect to ethanol conversion, although it is 100% in all cases, rather than ethylene conversion due to varying ethylene selectivities observed for upstream Ga-zeolites. The selectivities reported in FIG. 9A-FIG. 9D reflect the entire system (i.e., both catalyst beds). Within the first 5 h of reaction, there was a monotonic reduction in propylene selectivity (starting from an initial value of 30%) with a concomitant increase in C₄₌, the side-product commonly reported for reactions over CHA-type zeolites (Dai, W., Sun, X.; Tang, B.; Wu, G.; Li, L.; Guan, N.; Hunger, M., Verifying the mechanism of the ethene-to-propene conversion on zeolite H-SSZ-13. J. Catal. 2014, 314, 10-20). When the Ga-MCM-22 catalyst was placed upstream of the Al-SSZ-13 catalyst in a dual bed configuration (FIG. 9B), there was an increase in propylene selectivity (ca. 40%) and a decrease in the amount of C₄₌ side product.

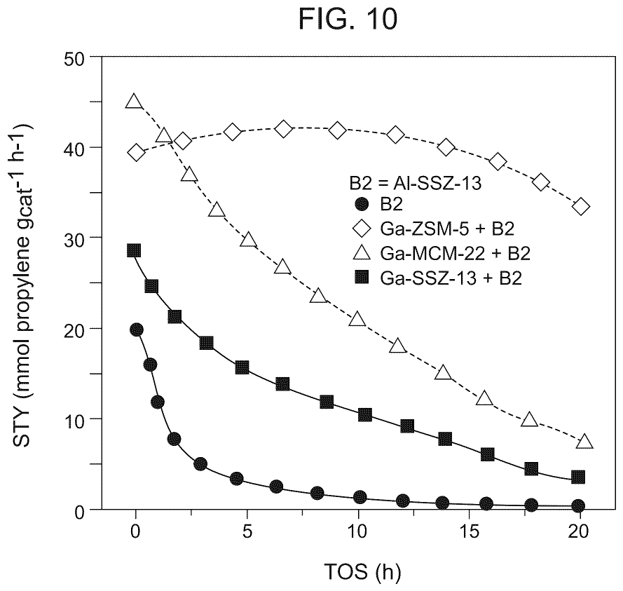

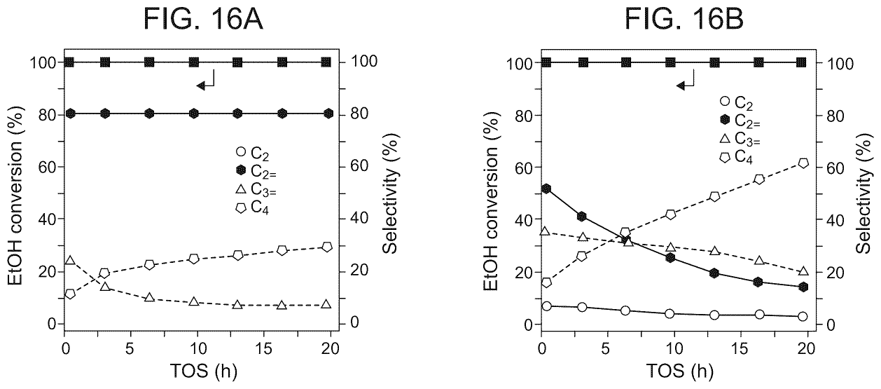

In the work described herein, we assessed two different dual bed configurations where the downstream catalyst was fixed (Al-SSZ-13) and we replaced the catalyst upstream with Ga-SSZ-13 (FIG. 9C) and Ga-ZSM-5 (FIG. 9D). The Ga-SSZ-13(B1)-Al-SSZ-13(B2) dual bed resulted in a higher propylene selectivity (ca. 70%) due to the generation of propylene in the upstream reaction over Ga-SSZ-13 (FIG. 16A). This was also reflected in a noticeable increase in propylene space time yield (STY, FIG. 10). The Ga-SSZ-13(B1)-Al-SSZ-13(B2) dual bed also produced a small amount of ethane (ca. 10%) as an additional side product. The Ga-ZSM-5(B1)-Al-SSZ-13(B2) dual bed (FIG. 9D) was the best performing configuration with the highest propylene selectivity (>50%) and lowest C₄-selectivity (<10%) over the 20 h reaction period. One unique feature of this dual bed configuration was a relatively constant selectivity profile with time on stream compared to the other three dual/single bed configurations. Without being bound by theory, this can be explained in part by the fact that Ga-ZSM-5 produces the highest amount of propylene upstream, which is also reflected in the highest propylene space time yield for the Ga-ZSM-5(B1)-Al-SSZ-13(B2) dual bed (FIG. 10).

In summary, we have shown that gallosilicate zeolites are superior alcohol dehydration catalysts relative to their aluminosilicate counterparts, with Ga-MCM-22 surprisingly outperforming all conventional dehydration catalysts reported in literature. In the methanol to DME reaction, Ga-MCM-22 exhibited nearly 100% methanol conversion and DME selectivity with a very slow rate of deactivation. The fact that Ga-SSZ-13 and Ga-ZSM-5 catalysts were less active and selective than Ga-MCM-22 suggests there are differences in the intrinsic strength (and possibly speciation) of Ga acid sites as a function of zeolite crystal structure. Differences in activity among the three Ga-zeolites was less evident in the ethanol dehydration reaction, but significant differences in product selectivity align with the results of methanol dehydration pointing to variable properties of Ga sites in gallosilicate zcolites.

The exact nature of active sites in Ga-zeolites remains elusive and is a subject of ongoing investigation. The literature attributes the reactivity of alcohol dehydration catalysts to Brønsted acid sites rather than Lewis acids, however, the best performing Ga-zeolite (Ga-MCM-22) contains a much higher percentage of Lewis acid sites relative to other Ga-zeolites examined in this study (Carr, R. T.; Neurock, M.; Iglesia, E., Catalytic consequences of acid strength in the conversion of methanol to dimethyl ether. J. Catal. 2011, 278 (1), 78-93; Bates, J. S.; Bukowski, B. C.; Greeley, J.; Gounder, R., Structure and solvation of confined water and water-ethanol clusters within microporous Brønsted acids and their effects on ethanol dehydration catalysis. Chem. Sci. Technol. 2020, 11 (27), 7102-7122; Park, J.; Cho, J.; Park, M.-J.; Lee, W. B., Microkinetic modeling of DME synthesis from methanol over H-zeolite catalyst: Associative vs. dissociative pathways. Catal. Today 2021, 375, 314-323). We confirmed that this performance is not entirely attributed to the presence of small amounts of extra-framework gallium oxide (FIG. 15), but it is less evident what role extra-framework Ga³⁺ species contribute to dehydration reactions. Our previous study using combined ⁷¹Ga MAS NMR and X-ray adsorption spectroscopy (XAS) revealed the challenges of quantifying the fractions of tetrahedral framework and extra-framework Ga species (Zhou, Y.; Thirumalai, H.; Smith, S. K.; Whitmire, K. H.; Liu, J.; Frenkel, A. I.; Grabow, L. C.; Rimer, J. D., Ethylene dehydroaromatization over Ga-ZSM-5 catalysts: nature and role of gallium speciation. Angew. Chem. Int. Ed. 2020, 59 (44), 19592-19601). In the previous study, we reported density functional theory calculations of Ga,Al-ZSM-5 catalysts for non-oxidative ethylene dehydroaromatization wherein it was observed that the barrier for ethylene activation over Ga Brønsted acid sites is nearly twice that of Al sites. Therefore, it is surprising that C₃ and C₄ products are formed during ethanol dehydration over some Ga-zeolites (e.g., Ga-SSZ-13 and Ga-ZSM-5) but not for others (e.g., Ga-MCM-22). Without being bound by theory, this seems to suggest there is a unique property of active sites in the Ga-MCM-22 catalyst that extends beyond shape selectivity.

One of the advantages of Ga-zeolites over conventional Al-zeolites for alcohol dehydration reactions is the ability to generate the catalyst via a one-pot (single-step) synthesis, thereby avoiding the need to perform post-synthesis functionalization, oxide impregnation, or ion exchange to reduce the acid site density. This enables reactions with Ga-zeolites to be carried out at much higher space velocity (i.e., less alcohol contact time) to achieve higher conversion and selectivity than what has been reported for other Al-zeolites and related materials. In the work described herein, we demonstrated the strategic advantages of using zeolites with reduced acid strength in tandem reactions wherein the Ga-zeolite is more desirable for downstream processes utilizing Al-zeolites to convert intermediates to light olefins. To the best of our knowledge, prior literature studies have not been reported for ethanol to propylene reactions over Al-zeolites. Another potential downside of using dual bed configurations in scaled-up reactors is increased pressure drop. However, for the conditions tested in our studies, both of these effects do not appear to compromise the overall performance of dual bed Ga-/Al-zeolite configurations, which outperform conventional single-bed Al-zeolite counterparts. Indeed, the Ga-MCM-22 catalyst exhibits the highest alcohol conversion and desired product selectivity among any alcohol dehydration catalyst reported in literature. Moreover, we demonstrate that the long lifetime of this catalyst is maintained at higher reaction temperatures, which addresses concerns raised in the literature regarding the need to develop more (hydro) thermally-stable zeolite dehydration catalysts (Fu, Y.; Hong, T.; Chen, J.; Auroux, A.; Shen, J., Surface acidity and the dehydration of methanol to dimethyl ether. Thermochim. Acta 2005, 434 (1-2), 22-26; Phung, T. K.; Hernández, L. P.; Lagazzo, A.; Busca, G., Dehydration of ethanol over zeolites, silica alumina and alumina: Lewis acidity, Brønsted acidity and confinement effects. Appl. Catal. A-Gen 2015, 493, 77-89). In the case of MTH reactions, we also show for the tandem reaction that the generation of DME reduces the rate of coking in Al-zeolites, thereby extending catalyst lifetime.

In general, in the literature zeolites with reduced acidity tend to be suboptimal catalysts for a majority of hydrocarbon processing reactions (e.g., cracking, MTH, oligomerization, etc.). In contrast to literature reports, in the work described herein, we show that Ga-zeolites are effective dehydration catalysts and can, therefore, be used in tandem with more acidic zeolites to enhance the production of light olefins. Although more in-depth studies are required to fully elucidate the fundamental mechanisms of these reactions and the nature of the active site(s), without being bound by theory we posit that Ga-zeolites could be used for the dehydration of other alcohols (i.e., beyond methanol and ethanol). To this end, we believe that the approach demonstrated in this study offers a guideline for engineering heteroatom zeolite isostructures with tailored acidity for a broader range of tandem reactions.

In various embodiments, the gallium zeolites of the present invention provide a significant improvement over current state-of-the-art dehydration catalysts. In various embodiments, the gallium zeolites of the present invention are hydrothermally stable, can operate at higher temperatures, and provide better conversion and selectivity than current state-of-the-art dehydration catalysts. For example, the Ga-MCM-22 catalyst of the present invention, in particular, outperformed current-state-of-the-art dehydration catalysts. In various embodiments, the gallium zeolites of the present invention improve alcohol dehydration reactions and enable a wider range of options for tandem catalysts to convert products into value-added chemicals (e.g., light olefins).

### Various Non-Limiting Embodiments of the Invention

Non-limiting embodiments include those listed below.

Embodiment 1. A zeolite comprising a microporous framework comprising a plurality of micropores, wherein the microporous framework comprises a framework type, and wherein the microporous framework comprises silicon atoms, oxygen atoms, and gallium atoms.

Embodiment 2. The zeolite of embodiment 1, further comprising at least one extra-framework species.

Embodiment 3. The zeolite of embodiment 2, wherein the at least one extra-framework species is at least one gallium species.

Embodiment 4. The zeolite of embodiment 3, wherein the at least one gallium species is at least one gallium oxide species.

Embodiment 5. The zeolite of embodiment 4, wherein the at least one gallium oxide species is Ga₂O₃.

Embodiment 6. The zeolite of any one of embodiments 2-5, wherein the at least one extra-framework species is dispersed throughout at least a portion of the plurality of micropores.

Embodiment 7. The zeolite of any one of embodiments 1-6, wherein the framework type is MWW, CHA, or MFI.

Embodiment 8. The zeolite of any one of embodiments 1-7, wherein the framework type is MWW.

Embodiment 9. The zeolite of any one of embodiments 1-8, wherein the zeolite does not comprise aluminum, aluminum atoms, aluminum species, or aluminum moieties.

Embodiment 10. The zeolite of any one of embodiments 1-9, wherein the microporous framework does not comprise aluminum, aluminum atoms, aluminum species, or aluminum moieties.

Embodiment 11. The zeolite of any one of embodiments 2-10, wherein the extra-framework species does not comprise aluminum, aluminum atoms, aluminum species, or aluminum moieties.

Embodiment 12. The zeolite of any one of embodiments 1-11, wherein the zeolite is a gallosilicate zeolite.

Embodiment 13. The zeolite of embodiment 12, wherein the gallosilicate zeolite is a Ga-MCM-22 zeolite, Ga-SSZ-13 zeolite, or Ga-ZSM-5 zeolite.

Embodiment 14. The zeolite of embodiment 12, wherein the gallosilicate zeolite is a Ga-MCM-22 zeolite and the framework type is MWW.

Embodiment 15. The zeolite of embodiment 12, wherein the gallosilicate zeolite is a Ga-SSZ-13 zeolite and the framework type is CHA.

Embodiment 16. The zeolite of embodiment 12, wherein the gallosilicate zeolite is a Ga-ZSM-5 zeolite and the framework type is MFI.

Embodiment 17. The zeolite of embodiment 12, wherein the gallosilicate zeolite is a Ga-MCM-22 zeolite.

Embodiment 18. The zeolite of any one of embodiments 1-11, wherein the zeolite is a Ga-MCM-22 zeolite, Ga-SSZ-13 zeolite, or Ga-ZSM-S zeolite.

Embodiment 19. The zeolite of any one of embodiments 1-11, wherein zeolite is a Ga-MCM-22 zeolite and the framework type is MWW.

Embodiment 20. The zeolite of any one of embodiments 1-11, wherein the zeolite is a Ga-SSZ-13 zeolite and the framework type is CHA.

Embodiment 21. The zeolite of any one of embodiments 1-11, wherein the zeolite is a Ga-ZSM-5 zeolite and the framework type is MFI.

Embodiment 22. The zeolite of any one of embodiments 1-11, wherein the zeolite is a Ga-MCM-22 zeolite.

Embodiment 23. A tandem catalyst, comprising: a first zeolite, wherein the first zeolite is a zeolite of any one of embodiments 1-22; and a second zeolite, wherein the first zeolite and the second zeolite are different from one another.

Embodiment 24. The tandem catalyst of embodiment 23, wherein the second zeolite is an aluminosilicate zeolite, and wherein the aluminosilicate zeolite comprises a framework type.

Embodiment 25. The tandem catalyst of embodiment 24, wherein the framework type is MWW, CHA, or MFI

Embodiment 26. The tandem catalyst of embodiment 24 or embodiment 25, wherein the aluminosilicate zeolite is a Al-MCM-22 zeolite, Al-SSZ-13 zeolite, or Al-ZSM-5 zeolite.

Embodiment 27. The tandem catalyst of embodiment 24 or embodiment 25, wherein the aluminosilicate zeolite is a Al-MCM-22 zeolite and the framework type is MWW.

Embodiment 28. The tandem catalyst of embodiment 24 or embodiment 25, wherein the aluminosilicate zeolite is a Al-SSZ-13 zeolite and the framework type is CHA.

Embodiment 29. The tandem catalyst of embodiment 24 or embodiment 25, wherein the aluminosilicate zeolite is a Al-ZSM-5 zeolite and the framework type is MFI.

Embodiment 30. A catalyst mixture, comprising: a first zeolite, wherein the first zeolite is a zeolite of any one of embodiments 1-22; and a second zeolite, wherein the first zeolite and the second zeolite are different from one another.

Embodiment 31. The catalyst mixture of embodiment 30, wherein the second zeolite is an aluminosilicate zeolite, and wherein the aluminosilicate zeolite comprises a framework type.

Embodiment 32. The catalyst mixture of embodiment 31, wherein the framework type is MWW, CHA, or MFL.

Embodiment 33. The catalyst mixture of embodiment 31 or embodiment 32, wherein the aluminosilicate zeolite is a Al-MCM-22 zeolite, Al-SSZ-13 zeolite, or Al-ZSM-5 zeolite.

Embodiment 34. The catalyst mixture of embodiment 31 or embodiment 32, wherein the aluminosilicate zeolite is a Al-MCM-22 zeolite and the framework type is MWW.

Embodiment 35. The catalyst mixture of embodiment 31 or embodiment 32, wherein the aluminosilicate zeolite is a Al-SSZ-13 zeolite and the framework type is CHA.

Embodiment 36. The catalyst mixture of embodiment 31 or embodiment 32, wherein the aluminosilicate zeolite is a Al-ZSM-S zeolite and the framework type is MFI.

Embodiment 37. A system, comprising: an inlet port; a reaction chamber, wherein the reaction chamber is in communication with the inlet port, wherein the reaction chamber contains a first zeolite, and wherein the first zeolite is a zeolite of any one of embodiments 1-22; and an outlet port, wherein the outlet port is in communication with the reaction chamber.

Embodiment 38. The system of embodiment 37, wherein the reaction chamber further contains a second zeolite, and wherein the first zeolite and the second zeolite are different from one another.

Embodiment 39. The system of embodiment 38, wherein the second zeolite is an aluminosilicate zeolite, and wherein the aluminosilicate zeolite comprises a framework type.

Embodiment 40. The system of embodiment 39, wherein the framework type is MWW, CHA, or MFI.

Embodiment 41. The system of embodiment 39 or embodiment 40, wherein the aluminosilicate zeolite is a Al-MCM-22 zeolite, Al-SSZ-13 zeolite, or Al-ZSM-5 zeolite.

Embodiment 42. The system of embodiment 39 or embodiment 40, wherein the aluminosilicate zeolite is a Al-MCM-22 zeolite and the framework type is MWW.

Embodiment 43. The system of embodiment 39 or embodiment 40, wherein the aluminosilicate zeolite is a Al-SSZ-13 zeolite and the framework type is CHA.

Embodiment 44. The system of embodiment 39 or embodiment 40, wherein the aluminosilicate zeolite is a Al-ZSM-5 zeolite and the framework type is MFI.

Embodiment 45. A system, comprising: an inlet port; a first reaction chamber, wherein the first reaction chamber is in communication with the inlet port, wherein the first reaction chamber contains a first zeolite, and wherein the first zeolite is a zeolite of any one of embodiments 1-22; a second reaction chamber, wherein the second reaction chamber is in communication with the first reaction chamber, wherein the second reaction chamber contains a second zeolite, and wherein the first zeolite and the second zeolite are different from one another; and an outlet port, wherein the outlet port is in communication with the second reaction chamber.

Embodiment 46. The system of embodiment 45, wherein the first reaction chamber is positioned upstream from the second reaction chamber.

Embodiment 47. The system of embodiment 45 or embodiment 46, wherein the second zeolite is an aluminosilicate zeolite, and wherein the aluminosilicate zeolite comprises a framework type.

Embodiment 48. The system of embodiment 47, wherein the framework type is MWW, CHA, or MFI.

Embodiment 49. The system of embodiment 47 or embodiment 48, wherein the aluminosilicate zeolite is a Al-MCM-22 zeolite, Al-SSZ-13 zeolite, or Al-ZSM-5 zeolite.

Embodiment 50. The system of embodiment 47 or embodiment 48, wherein the aluminosilicate zeolite is a Al-MCM-22 zeolite and the framework type is MWW.

Embodiment 51. The system of embodiment 47 or embodiment 48, wherein the aluminosilicate zeolite is a Al-SSZ-13 zeolite and the framework type is CHA.

Embodiment 52. The system of embodiment 47 or embodiment 48, wherein the aluminosilicate zeolite is a Al-ZSM-5 zeolite and the framework type is MFI.

Embodiment 53. A method for the dehydration of at least one alcohol to form at least one olefin, the method comprising: contacting a feedstock comprising at least one alcohol with a zeolite of any one of embodiments 1-22 to convert the at least one alcohol to at least one olefin.

Embodiment 54. The method of embodiment 53, wherein the feedstock is contacted with the zeolite under conditions effective to convert the at least one alcohol to the at least one olefin.

Embodiment 55. The method of embodiment 53 or embodiment 54, wherein the at least one alcohol is selected from the group consisting of methanol, ethanol, and combinations thereof.

Embodiment 56. The method of any one of embodiments 53-55, wherein the at least one olefin is selected from the group consisting of ethylene, propylene, and combinations thereof.

Embodiment 57. A method for the dehydration of at least one alcohol to form at least one olefin, the method comprising: contacting a feedstock comprising at least one alcohol with a first zeolite to form at least one intermediate compound, wherein the first zeolite is a zeolite of any one of embodiments 1-22; and contacting the at least one intermediate compound with a second zeolite to form at least one olefin, wherein the first zeolite and the second zeolite are different from one another.

Embodiment 58. The method of embodiment 57, wherein the second zeolite is an aluminosilicate zeolite, and wherein the aluminosilicate zeolite comprises a framework type.

Embodiment 59. The method of embodiment 58, wherein the framework type is MWW, CHA, or MFI.

Embodiment 60. The method of embodiment 58 or embodiment 59, wherein the aluminosilicate zeolite is a Al-MCM-22 zeolite, Al-SSZ-13 zeolite, or Al-ZSM-5 zeolite.

Embodiment 61. The method of embodiment 58 or embodiment 59, wherein the aluminosilicate zeolite is a Al-MCM-22 zeolite and the framework type is MWW.

Embodiment 62. The method of embodiment 58 or embodiment 59, wherein the aluminosilicate zeolite is a Al-SSZ-13 zeolite and the framework type is CHA.

Embodiment 63. The system of embodiment 58 or embodiment 59, wherein the aluminosilicate zeolite is a Al-ZSM-5 zeolite and the framework type is MFI.

Embodiment 64. The method of any one of embodiments 57-63, wherein the feedstock is contacted with the first zeolite under conditions effective to convert the at least one alcohol to the at least one intermediate compound.

Embodiment 65. The method of any one of embodiments 57-64, wherein the at least one intermediate compound is contacted with the second zeolite under conditions effective to convert the at least one intermediate compound to the at least one olefin.

Embodiment 66. The method of any one of embodiments 57-65, wherein the at least one alcohol is selected from the group consisting of methanol, ethanol, and combinations thereof.

Embodiment 67. The method of any one of embodiments 57-66, wherein the at least one intermediate compound is ethylene, dimethyl ether, and combinations thereof.

Embodiment 68. The method of any one of embodiments 57-67, wherein the at least one olefin is selected from the group consisting of ethylene, propylene, and combinations thereof.

Embodiment 69. Use of a zeolite of any one of embodiments 1-22 for converting at least one alcohol to at least one olefin.

Embodiment 70. Use of a tandem catalyst of any one of embodiments 23-29 for converting at least one alcohol to at least one olefin.

Embodiment 71. Use of a catalyst mixture of any one of embodiments 30-36 for converting at least one alcohol to at least one olefin.

Non-limiting embodiments include those listed below.

Embodiment 72. A method for converting at least one alcohol to at least one hydrocarbon product, the method comprising: contacting a feedstock comprising at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one hydrocarbon product, wherein the first zeolite and the second zeolite are different from one another.

Embodiment 73. The method of embodiment 72, wherein contacting the feedstock comprising at least one alcohol with the first zeolite is performed at a temperature of 180° C. to 600° C.

Embodiment 74. The method of embodiment 72, wherein contacting the feedstock comprising at least one alcohol with the first zeolite is performed at a weight-hourly space velocity (WHSV) of 2 h⁻¹ to 20 h⁻¹.

Embodiment 75. The method of embodiment 72, wherein contacting the at least one intermediate compound with the second zeolite is performed at a temperature of 180° C. to 600° C.

Embodiment 76. The method of embodiment 72, wherein contacting the at least one intermediate compound with the second zeolite is performed at a weight-hourly space velocity (WHSV) of 2 h⁻¹ to 20 h⁻¹.

Embodiment 77. The method of claim embodiment 72, wherein the first zeolite comprises at least one first extra-framework species, with the proviso that the at least one first extra-framework species does not comprise aluminum.

Embodiment 78. The method of embodiment 77, wherein the at least one first extra-framework species is Ga₂O₃.

Embodiment 79. The method of embodiment 72, wherein the first framework type is MWW, CHA, or MFI

Embodiment 80. The method of embodiment 72, wherein the first zeolite is Ga-MCM-22 zeolite, Ga-SSZ-13 zeolite, or Ga-ZSM-5 zeolite.

Embodiment 81. The method of embodiment 72, wherein the first zeolite is Ga-MCM-22 zeolite and the first framework type is MWW, or wherein the first zeolite is a Ga-SSZ-13 zeolite and the first framework type is CHA, or wherein the first zeolite is a Ga-ZSM-5 zeolite and the first framework type is MFL.

Embodiment 82. The method of embodiment 72, wherein the at least one alcohol is selected from the group consisting of methanol, ethanol, and combination thereof.

Embodiment 83. The method of embodiment 72, wherein the at least one product is at least one hydrocarbon.

Embodiment 84. The method of embodiment 83, wherein the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, or any combination thereof.

Embodiment 85. The method of embodiment 83, wherein the at least one hydrocarbon is at least one olefin, or at least one aromatic hydrocarbon, or both at least one olefin and at least one aromatic hydrocarbon.

Embodiment 86. The method of embodiment 85, wherein the at least one olefin is selected from the group consisting of ethylene, propylene, and combination thereof.

Embodiment 87. The method of embodiment 72, wherein the at least one intermediate compound is ethylene, dimethyl ether, water, or any combination thereof.

Embodiment 88. The method of embodiment 72, wherein the second zeolite is an aluminosilicate zeolite, and wherein the aluminosilicate zeolite comprises a second framework type.

Embodiment 89. The method of embodiment 88, wherein the second framework type is MWW, CHA, or MFI

Embodiment 90. The method of embodiment 88, wherein the aluminosilicate zeolite is Al-MCM-22 zeolite, Al-SSZ-13 zeolite, or Al-ZSM-5 zeolite.

Embodiment 91. The method of embodiment 88, wherein the aluminosilicate zeolite is Al-MCM-22 zeolite and the second framework type is MWW, or wherein the aluminosilicate zeolite is Al-SSZ-13 zeolite and the second framework type is CHA, or wherein the aluminosilicate zeolite is Al-ZSM-5 zeolite and the framework type is MFI.

Embodiment 92. A system for converting at least one alcohol to at least one hydrocarbon product, comprising: an inlet port; a first reaction chamber, wherein the first reaction chamber is in communication with the inlet port, wherein the first reaction chamber contains a first zeolite, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; a second reaction chamber, wherein the second reaction chamber is in communication with the first reaction chamber, wherein the second reaction chamber contains a second zeolite, and wherein the first zeolite and the second zeolite are different from one another, and an outlet port, wherein the outlet port is in communication with the second reaction chamber.

Embodiment 93. The system of embodiment 92, wherein the first reaction chamber is positioned upstream from the second reaction chamber.

Embodiment 94. The system of embodiment 92, wherein the first zeolite comprises at least one first extra-framework species, with the proviso that the at least one first extra-framework species does not comprise aluminum.

Embodiment 95. The system of embodiment 94, wherein the at least one first extra-framework species is Ga₂O₃.

Embodiment 96. The system of embodiment 92, wherein the first framework type is MWW, CHA, or MFI.

Embodiment 97. The system of embodiment 92, wherein the first zeolite is Ga-MCM-22 zeolite, Ga-SSZ-13 zeolite, or Ga-ZSM-5 zeolite.

Embodiment 98. The system of embodiment 92, wherein the first zeolite is Ga-MCM-22 zeolite and the first framework type is MWW.

Embodiment 99. The system of embodiment 92, wherein the first zeolite is a Ga-SSZ-13 zeolite and the first framework type is CHA.

Embodiment 100. The system of embodiment 92, wherein the first zeolite is a Ga-ZSM-5 zeolite and the first framework type is MFI.

Embodiment 101. The system of embodiment 92, wherein the first reaction chamber comprises a first heater, or a first cooler, or both a first heater and a first cooler.

Embodiment 102. The system of embodiment 92, wherein the first reaction chamber is configured to heat the first zeolite to a temperature of 180° C. to 600° C.

Embodiment 103. The system of embodiment 92, wherein the second reaction chamber comprises a second heater, or a second cooler, or both a second heater and a second cooler.

Embodiment 104. The system of embodiment 92, wherein the second reaction chamber is configured to heat the second zeolite to a temperature of 180° C. to 600° C.

Embodiment 105. The system of embodiment 92, wherein the second zeolite is an aluminosilicate zeolite, and wherein the aluminosilicate zeolite comprises a second framework type.

Embodiment 106. The system of embodiment 105, wherein the second framework type is MWW, CHA, or MFI.

Embodiment 107. The system of embodiment 105, wherein the aluminosilicate zeolite is Al-MCM-22 zeolite, Al-SSZ-13 zeolite, or Al-ZSM-5 zeolite.

Embodiment 108. The system of embodiment 105, wherein the aluminosilicate zeolite is Al-MCM-22 zeolite and the second framework type is MWW.

Embodiment 109. The system of embodiment 105, wherein the aluminosilicate zeolite is Al-SSZ-13 zeolite and the second framework type is CHA.

Embodiment 110. The system of embodiment 105, wherein the aluminosilicate zeolite is Al-ZSM-5 zeolite and the framework type is MFI.

Embodiment 111. A method for the dehydration of at least one alcohol, the method comprising: contacting a feedstock comprising at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

Embodiment 112. The method of embodiment 111, wherein contacting the feedstock comprising at least one alcohol with the at least one zeolite is performed at a temperature of 180° C. to 600° C.

Embodiment 113. The method of embodiment 111, wherein contacting the feedstock comprising at least one alcohol with the at least one zeolite is performed at a weight-hourly space velocity (WHSV) of 2 h⁻¹ to 20 h⁻¹.

Embodiment 114. The method of embodiment 111, wherein the at least one zeolite comprises at least one extra-framework species, with the proviso that the at least one extra-framework species does not comprise aluminum.

Embodiment 115. The method of embodiment 114, wherein the at least one extra-framework species is Ga₂O₃.

Embodiment 116. The method of embodiment 111, wherein the framework type is MWW, CHA, or MFI.

Embodiment 117. The method of embodiment 111, wherein the at least one zeolite is Ga-MCM-22 zeolite, Ga-SSZ-13 zeolite, or Ga-ZSM-5 zeolite.

Embodiment 118. The method of embodiment 111, wherein the at least one zeolite is Ga-MCM-22 zeolite and the first framework type is MWW.

Embodiment 119. The method of embodiment 111, wherein the at least one zeolite is a Ga-SSZ-13 zeolite and the first framework type is CHA.

Embodiment 120. The method of embodiment 111, wherein the at least one zeolite is a Ga-ZSM-5 zeolite and the first framework type is MFI.

Embodiment 121. The method of embodiment 111, wherein the at least one alcohol is selected from the group consisting of methanol, ethanol, and combination thereof.

Embodiment 122. The method of embodiment 111, wherein the at least one product is at least one hydrocarbon, dimethyl ether, water, or any combination thereof.

Embodiment 123. The method of embodiment 122, wherein the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, or any combination thereof.

Embodiment 124. The method of embodiment 122, wherein the at least one hydrocarbon is at least one olefin, or at least one aromatic hydrocarbon, or both at least one olefin and at least one aromatic hydrocarbon.

Embodiment 125. The method of embodiment 124, wherein the at least one olefin is selected from the group consisting of ethylene, propylene, and combination thereof.

Embodiment 126. A system for the dehydration of at least one alcohol, comprising: an inlet port; a reaction chamber, wherein the reaction chamber is in communication with the inlet port, wherein the reaction chamber contains at least one zeolite, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms; and an outlet port, wherein the outlet port is in communication with the reaction chamber.

Embodiment 127. The system of embodiment 126, wherein the at least one zeolite comprises at least one extra-framework species, with the proviso that the at least one extra-framework species does not comprise aluminum.

Embodiment 128. The system of embodiment 126, wherein the at least one extra-framework species is Ga₂O₃.

Embodiment 129. The system of embodiment 126, wherein the framework type is MWW, CHA, or MFI.

Embodiment 130. The system of embodiment 126, wherein the at least one zeolite is Ga-MCM-22 zeolite, Ga-SSZ-13 zeolite, or Ga-ZSM-5 zeolite.

Embodiment 131. The system of embodiment 126, wherein the at least one zeolite is Ga-MCM-22 zeolite and the framework type is MWW.

Embodiment 132. The system of embodiment 126, wherein the at least one zeolite is a Ga-SSZ-13 zeolite and the framework type is CHA.

Embodiment 133. The system of embodiment 126, wherein the at least one zeolite is a Ga-ZSM-5 zeolite and the framework type is MFI.

Embodiment 134. The system of embodiment 126, wherein the reaction chamber comprises a heater, or a cooler, or both a heater and a cooler.

Embodiment 135. The system of embodiment 126, wherein the reaction chamber is configured to heat the at least one zeolite to a temperature of 180° C. to 600° C.

Embodiment 136. A method for converting at least one alcohol to at least one hydrocarbon product, the method comprising: contacting a feedstock comprising at least one alcohol with a catalyst mixture to form at least one hydrocarbon product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

Embodiment 137. The method of embodiment 136, wherein contacting the feedstock comprising at least one alcohol with the catalyst mixture is performed at a temperature of 180° C. to 600° C.

Embodiment 138. The method of embodiment 136, wherein contacting the feedstock comprising at least one alcohol with the catalyst mixture is performed at a weight-hourly space velocity (WHSV) of 2 h⁻¹ to 20 h⁻¹.

Embodiment 139. The method of embodiment 136, wherein the first zeolite comprises at least one first extra-framework species, with the proviso that the at least one first extra-framework species does not comprise aluminum.

Embodiment 140. The method of embodiment 139, wherein the at least one first extra-framework species is Ga₂O₃.

Embodiment 141. The method of embodiment 136, wherein the first framework type is MWW, CHA, or MFI.

Embodiment 142. The method of embodiment 136, wherein the first zeolite is Ga-MCM-22 zeolite, Ga-SSZ-13 zeolite, or Ga-ZSM-5 zeolite.

Embodiment 143. The method of embodiment 136, wherein the first zeolite is Ga-MCM-22 zeolite and the first framework type is MWW, or wherein the first zeolite is a Ga-SSZ-13 zeolite and the first framework type is CHA, or wherein the first zeolite is a Ga-ZSM-5 zeolite and the first framework type is MFI.

Embodiment 144. The method of embodiment 136, wherein the at least one alcohol is selected from the group consisting of methanol, ethanol, and combination thereof.

Embodiment 145. The method of embodiment 136, wherein the at least one product is at least one hydrocarbon.

Embodiment 146. The method of embodiment 145, wherein the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, or any combination thereof.

Embodiment 147. The method of embodiment 145, wherein the at least one hydrocarbon is at least one olefin, or at least one aromatic hydrocarbon, or both at least one olefin and at least one aromatic hydrocarbon.

Embodiment 148. The method of embodiment 136, wherein the at least one olefin is selected from the group consisting of ethylene, propylene, and combination thereof.

Embodiment 149. The method of embodiment 136, wherein the second zeolite is an aluminosilicate zeolite, and wherein the aluminosilicate zeolite comprises a second framework type.

Embodiment 150. The method of embodiment 149, wherein the second framework type is MWW, CHA, or MFL

Embodiment 151. The method of embodiment 149, wherein the aluminosilicate zeolite is Al-MCM-22 zeolite, Al-SSZ-13 zeolite, or Al-ZSM-5 zeolite.

Embodiment 152. The method of embodiment 149, wherein the aluminosilicate zeolite is Al-MCM-22 zeolite and the second framework type is MWW, or wherein the aluminosilicate zeolite is Al-SSZ-13 zeolite and the second framework type is CHA, or wherein the aluminosilicate zeolite is Al-ZSM-5 zeolite and the framework type is MFI.

Embodiment 153. A system for converting at least one alcohol to at least one hydrocarbon product, comprising: an inlet port; a reaction chamber, wherein the reaction chamber is in communication with the inlet port, wherein the reaction chamber contains a catalyst mixture, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

Embodiment 154. The system of embodiment 153, wherein the first zeolite comprises at least one first extra-framework species, with the proviso that the at least one first extra-framework species does not comprise aluminum.

Embodiment 155. The system of embodiment 154, wherein the at least one first extra-framework species is Ga₂O₃.

Embodiment 156. The system of embodiment 153, wherein the first framework type is MWW, CHA, or MFI.

Embodiment 157. The system of embodiment 153, wherein the first zeolite is Ga-MCM-22 zeolite, Ga-SSZ-13 zeolite, or Ga-ZSM-5 zeolite.

Embodiment 158. The system of embodiment 153, wherein the first zeolite is Ga-MCM-22 zeolite and the first framework type is MWW.

Embodiment 159. The system of embodiment 153, wherein the first zeolite is a Ga-SSZ-13 zeolite and the first framework type is CHA.

Embodiment 160. The system of embodiment 153, wherein the first zeolite is a Ga-ZSM-5 zeolite and the first framework type is MFI.

Embodiment 161. The system of embodiment 153, wherein the reaction chamber comprises a heater, or a cooler, or both a heater and a cooler.

Embodiment 162. The system of embodiment 153, wherein the reaction chamber is configured to heat the catalyst mixture to a temperature of 180° C. to 600° C.

Embodiment 163. The system of embodiment 153, wherein the second zeolite is an aluminosilicate zeolite, and wherein the aluminosilicate zeolite comprises a second framework type.

Embodiment 164. The system of embodiment 163, wherein the second framework type is MWW, CHA, or MFL.

Embodiment 165. The system of embodiment 163, wherein the aluminosilicate zeolite is Al-MCM-22 zeolite, Al-SSZ-13 zeolite, or Al-ZSM-5 zeolite.

Embodiment 166. The system of embodiment 163, wherein the aluminosilicate zeolite is Al-MCM-22 zeolite and the second framework type is MWW.

Embodiment 167. The system of embodiment 163, wherein the aluminosilicate zeolite is Al-SSZ-13 zeolite and the second framework type is CHA.

Embodiment 168. The system of embodiment 163, wherein the aluminosilicate zeolite is Al-ZSM-5 zeolite and the framework type is MFI.

Non-limiting embodiments include those listed below.

In various embodiments, the present invention provides a zeolite comprising a framework, wherein the framework comprises a framework type, and wherein the framework comprises silicon atoms, oxygen atoms, and gallium atoms.

In various embodiments, the present invention provides a zeolite comprising a framework, wherein the framework comprises a framework type, and wherein the framework comprises silicon atoms, oxygen atoms, and gallium atoms, with the proviso that the framework does not comprise aluminum atoms.

In various embodiments, the present invention provides a zeolite comprising a microporous framework, wherein the microporous framework comprises a framework type, and wherein the microporous framework comprises silicon atoms, oxygen atoms, and gallium atoms.

In various embodiments, the present invention provides a zeolite comprising a microporous framework, wherein the microporous framework comprises a framework type, and wherein the microporous framework comprises silicon atoms, oxygen atoms, and gallium atoms, with the proviso that the framework does not comprise aluminum atoms.

In various embodiments, the present invention provides a zeolite comprising a microporous framework comprising a plurality of micropores, wherein the microporous framework comprises a framework type, and wherein the microporous framework comprises silicon atoms, oxygen atoms, and gallium atoms.

In various embodiments, the present invention provides a zeolite comprising a microporous framework comprising a plurality of micropores, wherein the microporous framework comprises a framework type, and wherein the microporous framework comprises silicon atoms, oxygen atoms, and gallium atoms, with the proviso that the microporous framework does not comprise aluminum atoms.

In some embodiments, the framework does not comprise aluminum, aluminum atoms, aluminum species, or aluminum moieties. In some embodiments, the framework does not comprise aluminum atoms. In some embodiments, the framework does not comprise aluminum.

In some embodiments, the framework is a microporous framework.

In some embodiments, the zeolite has a micropore volume of 0.11 cm³ to 0.23 cm³. In some embodiments, the zeolite has a micropore volume of 0.11 cm³. In some embodiments, the zeolite has a micropore volume of 0.17 cm³. In some embodiments, the zeolite has a micropore volume of 0.23 cm³.

In some embodiments, the plurality of micropores has a micropore volume of 0.11 cm³ to 0.23 cm³. In some embodiments, the plurality of micropores has a micropore volume of 0.11 cm³. In some embodiments, the plurality of micropores has a micropore volume of 0.17 cm³. In some embodiments, the plurality of micropores has a micropore volume of 0.23 cm³.

In some embodiments, the zeolite has a BET total surface area of 344 m²/g to 633 m²/g. In some embodiments, the zeolite has a BET total surface area of 344 m²/g. In some embodiments, the zeolite has a BET total surface area of 513 m²/g. In some embodiments, the zeolite has a BET total surface area of 633 m²/g.

In some embodiments, the zeolite has a BET external surface area of 71 m²/g to 109 m²/g. In some embodiments, the zeolite has a BET external surface area of 71 m²/g. In some embodiments, the zeolite has a BET external surface area of 88 m²/g. In some embodiments, the zeolite has a BET external surface area of 109 m²/g.

In some embodiments, the zeolite has a total acidity of 352 umol g⁻¹ to 633 umol g⁻¹. In some embodiments, the zeolite has a total acidity of 352 umol g⁻¹. In some embodiments, the zeolite has a total acidity of 491 umol g⁻¹. In some embodiments, the zeolite has a total acidity of 633 umol g⁻¹.

In some embodiments, the zeolite has a Bronsted acidity of 226 umol g⁻¹ to 506 umol g⁻¹. In some embodiments, the zeolite has a Bronsted acidity of 226 umol g⁻¹. In some embodiments, the zeolite has a Bronsted acidity of 281 umol g⁻¹. In some embodiments, the zeolite has a Bronsted acidity of 506 umol g⁻¹.

In some embodiments, the zeolite has a Lewis acidity of 71 umol g⁻¹ to 265 umol g⁻¹. In some embodiments, the zeolite has a Lewis acidity of 71 umol g⁻¹. In some embodiments, the zeolite has a Lewis acidity of 127 umol g⁻¹. In some embodiments, the zeolite has a Lewis acidity of 265 umol g⁻¹.

In some embodiments, the framework type is MWW, CHA, or MFI.

In some embodiments, the zeolite is Ga-MCM-22 zeolite, Ga-SSZ-13 zeolite, or Ga-ZSM-5 zeolite.

In some embodiments, the zeolite is a Ga-MCM-22 zeolite and the framework type is MWW. In some embodiments, the zeolite is a Ga-SSZ-13 zeolite and the framework type is CHA. In some embodiments, the zeolite is a Ga-ZSM-5 zeolite and the framework type is MFI.

In some embodiments, the zeolite comprises at least one extra-framework species.

In some embodiments, the zeolite comprises at least one extra-framework species, with the proviso that the at least one first extra-framework species does not comprise aluminum.

In some embodiments, the zeolite does not comprise aluminum, aluminum atoms, aluminum species, or aluminum moieties.

In some embodiments, the framework does not comprise aluminum, aluminum atoms, aluminum species, or aluminum moieties.

In some embodiments, the microporous framework does not comprise aluminum, aluminum atoms, aluminum species, or aluminum moieties.

In some embodiments, the extra-framework species does not comprise aluminum, aluminum atoms, aluminum species, or aluminum moieties.

In some embodiments, the zeolite is a gallium zeolite. In some embodiments, the zeolite is a gallosilicate zeolite. In some embodiments, the zeolite of the present invention is a gallosilicate zeolite. In some embodiments, the zeolite of the present invention is a gallium zeolite.

In some embodiments, the zeolite is a first zeolite. In some embodiments, the first zeolite is a gallium zeolite. In some embodiments, the first zeolite is a gallosilicate zeolite.

In some embodiments, the second zeolite is an aluminosilicate zeolite, and wherein the aluminosilicate zeolite comprises a second framework type.

In some embodiments, the second framework type is MWW, CHA, or MFI.

In some embodiments, the aluminosilicate zeolite is Al-MCM-22 zeolite, Al-SSZ-13 zeolite, or Al-ZSM-5 zeolite.

In some embodiments, the aluminosilicate zeolite is Al-MCM-22 zeolite and the second framework type is MWW, or wherein the aluminosilicate zeolite is Al-SSZ-13 zeolite and the second framework type is CHA, or wherein the aluminosilicate zeolite is Al-ZSM-5 zeolite and the framework type is MFI.

In some embodiments, the aluminosilicate zeolite has a micropore volume of 0.11 cm³ to 0.24 cm³. In some embodiments, the aluminosilicate zeolite has a micropore volume of 0.11 cm³. In some embodiments, the aluminosilicate zeolite has a micropore volume of 0.19 cm³. In some embodiments, the aluminosilicate zeolite has a micropore volume of 0.24 cm³.

In some embodiments, the aluminosilicate zeolite has a BET total surface area of 385 m²/g to 655 m²/g. In some embodiments, the aluminosilicate zeolite has a BET total surface area of 385 m²/g. In some embodiments, the aluminosilicate zeolite has a BET total surface area of 634 m²/g. In some embodiments, the aluminosilicate zeolite has a BET total surface area of 655 m²/g.

In some embodiments, the aluminosilicate zeolite has a BET external surface area of 60 m²/g to 149 m²/g. In some embodiments, the aluminosilicate zeolite has a BET external surface area of 60 m²/g. In some embodiments, the aluminosilicate zeolite has a BET external surface area of 97 m²/g. In some embodiments, the aluminosilicate zeolite has a BET external surface area of 149 m²/g.

In some embodiments, the aluminosilicate zeolite has a total acidity of 441 umol g⁻¹ to 666 umol g⁻¹. In some embodiments, the aluminosilicate zeolite has a total acidity of 441 umol g⁻¹. In some embodiments, the aluminosilicate zeolite has a total acidity of 485 umol g⁻¹ In some embodiments, the aluminosilicate zeolite has a total acidity of 666 umol g⁻¹.

In some embodiments, the aluminosilicate zeolite has a Bronsted acidity of 370 umol g⁻¹ to 612 umol g⁻¹. In some embodiments, the aluminosilicate zeolite has a Bronsted acidity of 370 umol g⁻¹. In some embodiments, the aluminosilicate zeolite has a Bronsted acidity of 412 umol g⁻¹. In some embodiments, the aluminosilicate zeolite has a Bronsted acidity of 612 umol g⁻¹.

In some embodiments, the aluminosilicate zeolite has a Lewis acidity of 54 umol g⁻¹ to 73 umol g⁻¹. In some embodiments, the aluminosilicate zeolite has a Lewis acidity of 54 umol g⁻¹. In some embodiments, the aluminosilicate zeolite has a Lewis acidity of 71 umol g⁻¹. In some embodiments, the aluminosilicate zeolite has a Lewis acidity of 73 umol g⁻¹.

In various embodiments, the present invention provides a method for the dehydration of at least one alcohol, the method comprising: contacting a feedstock comprising at least one alcohol with a first zeolite to form at least one intermediate compound, wherein the first zeolite is a zeolite of the present invention; and contacting the at least one intermediate compound with a second zeolite to form at least one product, wherein the first zeolite and the second zeolite are different from one another.

In various embodiments, the present invention provides a method for the dehydration of at least one alcohol, the method comprising: contacting at least one alcohol with a first zeolite to form at least one intermediate compound, wherein the first zeolite is a zeolite of the present invention; and contacting the at least one intermediate compound with a second zeolite to form at least one product, wherein the first zeolite and the second zeolite are different from one another.

In various embodiments, the present invention provides a method for the dehydration of at least one alcohol, the method comprising: contacting a feedstock comprising at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one product, wherein the first zeolite and the second zeolite are different from one another.

In various embodiments, the present invention provides a method for the dehydration of at least one alcohol, the method comprising: contacting at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one product, wherein the first zeolite and the second zeolite are different from one another.

In various embodiments, the present invention provides a method for converting at least one alcohol to at least one hydrocarbon product, the method comprising: contacting a feedstock comprising at least one alcohol with a first zeolite to form at least one intermediate compound, wherein the first zeolite is a zeolite of the present invention; and contacting the at least one intermediate compound with a second zeolite to form at least one hydrocarbon product, wherein the first zeolite and the second zeolite are different from one another.

In various embodiments, the present invention provides a method for converting at least one alcohol to at least one product, the method comprising: contacting a feedstock comprising at least one alcohol with a first zeolite to form at least one intermediate compound, wherein the first zeolite is a zeolite of the present invention; and contacting the at least one intermediate compound with a second zeolite to form at least one product, wherein the first zeolite and the second zeolite are different from one another.

In various embodiments, the present invention provides a method for converting at least one alcohol to at least one hydrocarbon product, the method comprising: contacting at least one alcohol with a first zeolite to form at least one intermediate compound, wherein the first zeolite is a zeolite of the present invention; and contacting the at least one intermediate compound with a second zeolite to form at least one hydrocarbon product, wherein the first zeolite and the second zeolite are different from one another.

In various embodiments, the present invention provides a method for converting at least one alcohol to at least one product, the method comprising: contacting at least one alcohol with a first zeolite to form at least one intermediate compound, wherein the first zeolite is a zeolite of the present invention; and contacting the at least one intermediate compound with a second zeolite to form at least one product, wherein the first zeolite and the second zeolite are different from one another.

In various embodiments, the present invention provides a method for converting at least one alcohol to at least one hydrocarbon product, the method comprising: contacting a feedstock comprising at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one hydrocarbon product, wherein the first zeolite and the second zeolite are different from one another.

In various embodiments, the present invention provides a method for converting at least one alcohol to at least one product, the method comprising: contacting a feedstock comprising at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one product, wherein the first zeolite and the second zeolite are different from one another.

In various embodiments, the present invention provides a method for converting at least one alcohol to at least one hydrocarbon product, the method comprising: contacting at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one hydrocarbon product, wherein the first zeolite and the second zeolite are different from one another.

In various embodiments, the present invention provides a method for converting at least one alcohol to at least one product, the method comprising: contacting at least one alcohol with a first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with a second zeolite to form at least one product, wherein the first zeolite and the second zeolite are different from one another.

In various embodiments, the present invention provides a method for the dehydration of at least one alcohol, the method comprising: providing a feedstock comprising at least one alcohol; providing a first zeolite; providing a second zeolite; contacting the feedstock comprising at least one alcohol with the first zeolite to form at least one intermediate compound, wherein the first zeolite is a zeolite of the present invention; and contacting the at least one intermediate compound with the second zeolite to form at least one product, wherein the first zeolite and the second zeolite are different from one another.

In various embodiments, the present invention provides a method for the dehydration of at least one alcohol, the method comprising: providing at least one alcohol; providing a first zeolite; providing a second zeolite; contacting the at least one alcohol with the first zeolite to form at least one intermediate compound, wherein the first zeolite is a zeolite of the present invention; and contacting the at least one intermediate compound with the second zeolite to form at least one product, wherein the first zeolite and the second zeolite are different from one another.

In various embodiments, the present invention provides a method for the dehydration of at least one alcohol, the method comprising: providing a feedstock comprising at least one alcohol; providing a first zeolite; providing a second zeolite; contacting the feedstock comprising at least one alcohol with the first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with the second zeolite to form at least one product, wherein the first zeolite and the second zeolite are different from one another.

In various embodiments, the present invention provides a method for the dehydration of at least one alcohol, the method comprising: providing at least one alcohol; providing a first zeolite; providing a second zeolite; contacting at least one alcohol with the first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with the second zeolite to form at least one product, wherein the first zeolite and the second zeolite are different from one another.

In various embodiments, the present invention provides a method for converting at least one alcohol to at least one hydrocarbon product, the method comprising: providing a feedstock comprising at least one alcohol; providing a first zeolite; providing a second zeolite; contacting the feedstock comprising at least one alcohol with the first zeolite to form at least one intermediate compound, wherein the first zeolite is a zeolite of the present invention; and contacting the at least one intermediate compound with the second zeolite to form at least one hydrocarbon product, wherein the first zeolite and the second zeolite are different from one another.

In various embodiments, the present invention provides a method for converting at least one alcohol to at least one product, the method comprising: providing a feedstock comprising at least one alcohol; providing a first zeolite; providing a second zeolite; contacting the feedstock comprising at least one alcohol with a first zeolite to form at least one intermediate compound, wherein the first zeolite is a zeolite of the present invention; and contacting the at least one intermediate compound with the second zeolite to form at least one product, wherein the first zeolite and the second zeolite are different from one another.

In various embodiments, the present invention provides a method for converting at least one alcohol to at least one hydrocarbon product, the method comprising: providing at least one alcohol; providing a first zeolite; providing a second zeolite; contacting the at least one alcohol with the first zeolite to form at least one intermediate compound, wherein the first zeolite is a zeolite of the present invention; and contacting the at least one intermediate compound with the second zeolite to form at least one hydrocarbon product, wherein the first zeolite and the second zeolite are different from one another.

In various embodiments, the present invention provides a method for converting at least one alcohol to at least one product, the method comprising: providing at least one alcohol; providing a first zeolite; providing a second zeolite; contacting the at least one alcohol with the first zeolite to form at least one intermediate compound, wherein the first zeolite is a zeolite of the present invention; and contacting the at least one intermediate compound with the second zeolite to form at least one product, wherein the first zeolite and the second zeolite are different from one another.

In various embodiments, the present invention provides a method for converting at least one alcohol to at least one hydrocarbon product, the method comprising: providing a feedstock comprising at least one alcohol; providing a first zeolite; providing a second zeolite; contacting the feedstock comprising at least one alcohol with the first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with the second zeolite to form at least one hydrocarbon product, wherein the first zeolite and the second zeolite are different from one another.

In various embodiments, the present invention provides a method for converting at least one alcohol to at least one product, the method comprising: providing a feedstock comprising at least one alcohol; providing a first zeolite; providing a second zeolite; contacting the feedstock comprising at least one alcohol with the first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with the second zeolite to form at least one product, wherein the first zeolite and the second zeolite are different from one another.

In various embodiments, the present invention provides a method for converting at least one alcohol to at least one hydrocarbon product, the method comprising: providing at least one alcohol; providing a first zeolite; providing a second zeolite; contacting at least one alcohol with the first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with the second zeolite to form at least one hydrocarbon product, wherein the first zeolite and the second zeolite are different from one another.

In various embodiments, the present invention provides a method for converting at least one alcohol to at least one product, the method comprising: providing at least one alcohol; providing a first zeolite; providing a second zeolite; contacting at least one alcohol with the first zeolite to form at least one intermediate compound, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and contacting the at least one intermediate compound with the second zeolite to form at least one product, wherein the first zeolite and the second zeolite are different from one another.

In some embodiments, the first zeolite does not comprise aluminum, aluminum atoms, aluminum species, or aluminum moieties.

In some embodiments, the first microporous framework does not comprise aluminum, aluminum atoms, aluminum species, or aluminum moieties.

In some embodiments, the extra-framework species does not comprise aluminum, aluminum atoms, aluminum species, or aluminum moieties.

In some embodiments, contacting the feedstock comprising at least one alcohol with the first zeolite to form at least one intermediate compound is performed under conditions effective to form the at least one intermediate compound.

In some embodiments, contacting the at least one intermediate compound with a second zeolite to form at least one hydrocarbon product is performed under conditions effective to form the at least one hydrocarbon product.

In some embodiments, the first zeolite and the second zeolite are not in physical contact with one another. In some embodiments, the first zeolite and the second zeolite are separated from one another.

In some embodiments, contacting the feedstock comprising at least one alcohol with the first zeolite is performed at a temperature of 180° C. to 600° C.

In some embodiments, contacting the feedstock comprising at least one alcohol with the first zeolite is performed at a weight-hourly space velocity (WHSV) of 2 h⁻¹ to 20 h⁻¹.

In some embodiments, contacting the at least one intermediate compound with the second zeolite is performed at a temperature of 180° C. to 600° C.

In some embodiments, contacting the at least one intermediate compound with the second zeolite is performed at a weight-hourly space velocity (WHSV) of 2 h⁻¹ to 20 h⁻¹.

In some embodiments, the first zeolite comprises at least one first extra-framework species. In some embodiments, the first zeolite comprises at least one first extra-framework species, with the proviso that the at least one first extra-framework species does not comprise aluminum.

In some embodiments, the at least one first extra-framework species is Ga₂O₃.

In some embodiments, the first framework type is MWW, CHA, or MFI.

In some embodiments, the first zeolite is Ga-MCM-22 zeolite, Ga-SSZ-13 zeolite, or Ga-ZSM-5 zeolite.

In some embodiments, the first zeolite is Ga-MCM-22 zeolite and the first framework type is MWW, or wherein the first zeolite is a Ga-SSZ-13 zeolite and the first framework type is CHA, or wherein the first zeolite is a Ga-ZSM-5 zeolite and the first framework type is MFI.

In some embodiments, the at least one alcohol is selected from the group consisting of methanol, ethanol, and combination thereof.

In some embodiments, the at least one product is at least one hydrocarbon.

In some embodiments, the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, or any combination thereof.

In some embodiments, the at least one hydrocarbon is at least one olefin, or at least one aromatic hydrocarbon, or both at least one olefin and at least one aromatic hydrocarbon.

In some embodiments, the at least one olefin is selected from the group consisting of ethylene, propylene, and combination thereof.

In some embodiments, the at least one intermediate compound is ethylene, dimethyl ether, water, or any combination thereof.

In some embodiments, the second zeolite is an aluminosilicate zeolite, and wherein the aluminosilicate zeolite comprises a second framework type.

In some embodiments, the second framework type is MWW, CHA, or MFL.

In some embodiments, the aluminosilicate zeolite is Al-MCM-22 zeolite, Al-SSZ-13 zeolite, or Al-ZSM-5 zeolite.

In some embodiments, the aluminosilicate zeolite is Al-MCM-22 zeolite and the second framework type is MWW, or wherein the aluminosilicate zeolite is Al-SSZ-13 zeolite and the second framework type is CHA, or wherein the aluminosilicate zeolite is Al-ZSM-5 zeolite and the framework type is MFI.

In some embodiments, the at least one intermediate compound is at least one first product. In some embodiments, the at least one intermediate compound is at least one intermediate product. In some embodiments, the at least one intermediate compound is at least one first hydrocarbon product. In some embodiments, the at least one product is at least one second product.

In various embodiments, the present invention provides a system for the dehydration of at least one alcohol, comprising: an inlet port; a first reaction chamber, wherein the first reaction chamber is in communication with the inlet port, wherein the first reaction chamber contains a first zeolite, wherein the first zeolite is a zeolite of the present invention; a second reaction chamber, wherein the second reaction chamber is in communication with the first reaction chamber, wherein the second reaction chamber contains a second zeolite, and wherein the first zeolite and the second zeolite are different from one another; and an outlet port, wherein the outlet port is in communication with the second reaction chamber.

In various embodiments, the present invention provides a system for the dehydration of at least one alcohol, comprising: an inlet port; a first reaction chamber, wherein the first reaction chamber is in communication with the inlet port, wherein the first reaction chamber contains a first zeolite, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; a second reaction chamber, wherein the second reaction chamber is in communication with the first reaction chamber, wherein the second reaction chamber contains a second zeolite, and wherein the first zeolite and the second zeolite are different from one another; and an outlet port, wherein the outlet port is in communication with the second reaction chamber.

In various embodiments, the present invention provides a system for converting at least one alcohol to at least one hydrocarbon product, comprising: an inlet port; a first reaction chamber, wherein the first reaction chamber is in communication with the inlet port, wherein the first reaction chamber contains a first zeolite, wherein the first zeolite is a zeolite of the present invention; a second reaction chamber, wherein the second reaction chamber is in communication with the first reaction chamber, wherein the second reaction chamber contains a second zeolite, and wherein the first zeolite and the second zeolite are different from one another; and an outlet port, wherein the outlet port is in communication with the second reaction chamber.

In various embodiments, the present invention provides a system for converting at least one alcohol to at least one hydrocarbon product, comprising: an inlet port; a first reaction chamber, wherein the first reaction chamber is in communication with the inlet port, wherein the first reaction chamber contains a first zeolite, wherein the first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; a second reaction chamber, wherein the second reaction chamber is in communication with the first reaction chamber, wherein the second reaction chamber contains a second zeolite, and wherein the first zeolite and the second zeolite are different from one another; and an outlet port, wherein the outlet port is in communication with the second reaction chamber.

In some embodiments, the first reaction chamber is positioned upstream from the second reaction chamber.

In some embodiments, the first zeolite comprises at least one first extra-framework species. In some embodiments, the first zeolite comprises at least one first extra-framework species, with the proviso that the at least one first extra-framework species does not comprise aluminum. In some embodiments, the at least one first extra-framework species is Ga₂O₃.

In some embodiments, the first framework type is MWW, CHA, or MFI.

In some embodiments, the first zeolite is Ga-MCM-22 zeolite, Ga-SSZ-13 zeolite, or Ga-ZSM-5 zeolite.

In some embodiments, the first zeolite is Ga-MCM-22 zeolite and the first framework type is MWW.

In some embodiments, the first zeolite is a Ga-SSZ-13 zeolite and the first framework type is CHA.

In some embodiments, the first zeolite is a Ga-ZSM-5 zeolite and the first framework type is MFI.

In some embodiments, the first reaction chamber comprises a first heater, or a first cooler, or both a first heater and a first cooler.

In some embodiments, the first reaction chamber is configured to heat the first zeolite to a temperature of 180° C. to 600° C.

In some embodiments, the first reaction chamber is configured to contact the feedstock comprising at least one alcohol with the first zeolite at a weight-hourly space velocity (WHSV) of 2 h⁻¹ to 20 h⁻¹. In some embodiments, the first reaction chamber is configured to contact the at least one alcohol with the first zeolite at a weight-hourly space velocity (WHSV) of 2 h⁻¹ to 20 h⁻¹.

In some embodiments, the second reaction chamber comprises a second heater, or a second cooler, or both a second heater and a second cooler.

In some embodiments, the second reaction chamber is configured to beat the second zeolite to a temperature of 180° C. to 600° C.

In some embodiments, the second reaction chamber is configured to contact the intermediate compound with the second zeolite at a weight-hourly space velocity (WHSV) of 2 h⁻¹ to 20 h⁻¹.

In some embodiments, the second zeolite is an aluminosilicate zeolite, and wherein the aluminosilicate zeolite comprises a second framework type.

In some embodiments, the second framework type is MWW, CHA, or MFI.

In some embodiments, the aluminosilicate zeolite is Al-MCM-22 zeolite, Al-SSZ-13 zeolite, or Al-ZSM-S zeolite.

In some embodiments, the aluminosilicate zeolite is Al-MCM-22 zeolite and the second framework type is MWW.

In some embodiments, the aluminosilicate zeolite is Al-SSZ-13 zeolite and the second framework type is CHA.

In some embodiments, the aluminosilicate zeolite is Al-ZSM-5 zeolite and the framework type is MFI.

In various embodiments, the present invention provides a method for the dehydration of at least one alcohol, the method comprising: contacting a feedstock comprising at least one alcohol with at least one zeolite to form at least one product, wherein the at least one zeolite is a zeolite of the present invention.

In various embodiments, the present invention provides a method for the dehydration of at least one alcohol, the method comprising: contacting a feedstock with at least one zeolite to form at least one product, wherein the feedstock comprises at least one alcohol, and wherein the at least one zeolite is a zeolite of the present invention.

In various embodiments, the present invention provides a method for the dehydration of at least one alcohol, the method comprising: contacting at least one alcohol with at least one zeolite to form at least one product, wherein the at least one zeolite is a zeolite of the present invention.

In various embodiments, the present invention provides a method for the dehydration of at least one alcohol, the method comprising: contacting a feedstock comprising at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

In various embodiments, the present invention provides a method for the dehydration of at least one alcohol, the method comprising: contacting a feedstock with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms, wherein the feedstock comprises at least one alcohol.

In various embodiments, the present invention provides a method for the dehydration of at least one alcohol, the method comprising: contacting at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

In various embodiments, the present invention provides a method for the dehydration of at least one alcohol, the method comprising: providing a feedstock comprising at least one alcohol; providing at least one zeolite; contacting the feedstock comprising at least one alcohol with the at least one zeolite to form at least one product, wherein the at least one zeolite is a zeolite of the present invention.

In various embodiments, the present invention provides a method for the dehydration of at least one alcohol, the method comprising: providing a feedstock comprising at least one alcohol; providing at least one zeolite; contacting the feedstock with the at least one zeolite to form at least one product, wherein the feedstock comprises at least one alcohol, and wherein the at least one zeolite is a zeolite of the present invention.

In various embodiments, the present invention provides a method for the dehydration of at least one alcohol, the method comprising: providing at least one alcohol; providing at least one zeolite; contacting the at least one alcohol with the at least one zeolite to form at least one product, wherein the at least one zeolite is a zeolite of the present invention.

In various embodiments, the present invention provides a method for the dehydration of at least one alcohol, the method comprising: providing a feedstock comprising at least one alcohol; providing at least one zeolite; contacting the feedstock comprising at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

In various embodiments, the present invention provides a method for the dehydration of at least one alcohol, the method comprising: providing a feedstock comprising at least one alcohol; providing at least one zeolite; contacting the feedstock with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms, wherein the feedstock comprises at least one alcohol.

In various embodiments, the present invention provides a method for the dehydration of at least one alcohol, the method comprising: providing at least one alcohol; providing at least one zeolite; contacting the at least one alcohol with the at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

In some embodiments, contacting the feedstock comprising at least one alcohol with the at least one zeolite to form at least one product is performed under conditions effective to form the at least one product.

In some embodiments, contacting the at least one alcohol with the at least one zeolite to form at least one product is performed under conditions effective to form the at least one product.

In some embodiments, contacting the feedstock comprising at least one alcohol with the at least one zeolite is performed at a temperature of 180° C. to 600° C.

In some embodiments, contacting the feedstock comprising at least one alcohol with the at least one zeolite is performed at a weight-hourly space velocity (WHSV) of 2 h⁻¹ to 20 h⁻¹.

In some embodiments, the at least one zeolite comprises at least one extra-framework species. In some embodiments, the at least one zeolite comprises at least one extra-framework species, with the proviso that the at least one extra-framework species does not comprise aluminum. In some embodiments, the at least one extra-framework species is Ga₂O₃.

In some embodiments, the framework type is MWW, CHA, or MFI.

In some embodiments, the at least one zeolite is Ga-MCM-22 zeolite, Ga-SSZ-13 zeolite, or Ga-ZSM-5 zeolite.

In some embodiments, the at least one zeolite is Ga-MCM-22 zeolite and the first framework type is MWW.

In some embodiments, the at least one zeolite is a Ga-SSZ-13 zeolite and the first framework type is CHA.

In some embodiments, the at least one zeolite is a Ga-ZSM-5 zeolite and the first framework type is MFI.

In some embodiments, the at least one alcohol is selected from the group consisting of methanol, ethanol, and combination thereof.

In some embodiments, the at least one product is at least one hydrocarbon, dimethyl ether, water, or any combination thereof.

In some embodiments, the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, or any combination thereof.

In some embodiments, the at least one hydrocarbon is at least one olefin, or at least one aromatic hydrocarbon, or both at least one olefin and at least one aromatic hydrocarbon.

In some embodiments, the at least one olefin is selected from the group consisting of ethylene, propylene, and combination thereof.

In some embodiments, contacting the feedstock comprising at least one alcohol with the at least one zeolite to form at least one product is performed under conditions effective to form the at least one product.

In some embodiments, contacting the at least one alcohol with the at least one zeolite to form at least one product is performed under conditions effective to form the at least one product.

In various embodiments, the present invention provides a system for the dehydration of at least one alcohol, comprising: an inlet port; a reaction chamber, wherein the reaction chamber is in communication with the inlet port, wherein the reaction chamber contains at least one zeolite, wherein the at least one zeolite is a zeolite of the present invention; and an outlet port, wherein the outlet port is in communication with the reaction chamber.

In various embodiments, the present invention provides a system for the dehydration of at least one alcohol, comprising: an inlet port; a reaction chamber, wherein the reaction chamber is in communication with the inlet port, wherein the reaction chamber contains at least one zeolite, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms; and an outlet port, wherein the outlet port is in communication with the reaction chamber.

In some embodiments, the at least one zeolite comprises at least one extra-framework species, with the proviso that the at least one extra-framework species does not comprise aluminum.

In some embodiments, the at least one extra-framework species is Ga₂O₃.

In some embodiments, the framework type is MWW, CHA, or MFL

In some embodiments, the at least one zeolite is Ga-MCM-22 zeolite, Ga-SSZ-13 zeolite, or Ga-ZSM-5 zeolite.

In some embodiments, the at least one zeolite is Ga-MCM-22 zeolite and the framework type is MWW.

In some embodiments, the at least one zeolite is a Ga-SSZ-13 zeolite and the framework type is CHA.

In some embodiments, the at least one zeolite is a Ga-ZSM-5 zeolite and the framework type is MFI.

In some embodiments, the reaction chamber comprises a heater, or a cooler, or both a heater and a cooler.

In some embodiments, the reaction chamber is configured to heat the at least one zeolite to a temperature of 180° C. to 600° C.

In some embodiments, the reaction chamber is configured to contact the feedstock comprising at least one alcohol with the at least one zeolite at a weight-hourly space velocity (WHSV) of 2 h⁻¹ to 20 h⁻¹. In some embodiments, the reaction chamber is configured to contact the at least one alcohol with the at least one zeolite at a weight-hourly space velocity (WHSV) of 2 h⁻¹ to 20 h⁻¹.

In various embodiments, the present invention provides a method for the dehydration of at least one alcohol, the method comprising: contacting a feedstock comprising at least one alcohol with a catalyst mixture to form at least one product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for the dehydration of at least one alcohol, the method comprising: contacting at least one alcohol with a catalyst mixture to form at least one product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for the dehydration of at least one alcohol, the method comprising: contacting a feedstock comprising at least one alcohol with a catalyst mixture to form at least one product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a method for the dehydration of at least one alcohol, the method comprising: contacting at least one alcohol with a catalyst mixture to form at least one product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a method for converting at least one alcohol to at least one hydrocarbon product, the method comprising: contacting a feedstock comprising at least one alcohol with a catalyst mixture to form at least one hydrocarbon product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for converting at least one alcohol to at least one hydrocarbon product, the method comprising: contacting at least one alcohol with a catalyst mixture to form at least one hydrocarbon product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for converting at least one alcohol to at least one hydrocarbon product, the method comprising: contacting a feedstock comprising at least one alcohol with a catalyst mixture to form at least one hydrocarbon product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a method for converting at least one alcohol to at least one hydrocarbon product, the method comprising: contacting at least one alcohol with a catalyst mixture to form at least one hydrocarbon product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a method for the dehydration of at least one alcohol, the method comprising: providing a feedstock comprising at least one alcohol; providing a catalyst mixture; contacting the feedstock comprising at least one alcohol with the catalyst mixture to form at least one product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for the dehydration of at least one alcohol, the method comprising: providing at least one alcohol; providing at least one catalyst mixture; contacting at least one alcohol with a catalyst mixture to form at least one product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for the dehydration of at least one alcohol, the method comprising: providing a feedstock comprising at least one alcohol; providing a catalyst mixture; contacting the feedstock comprising at least one alcohol with the catalyst mixture to form at least one product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a method for the dehydration of at least one alcohol, the method comprising: providing at least one alcohol; providing contacting at least one alcohol with a catalyst mixture to form at least one product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms, and wherein the at least one first zeolite and the at least one second zeolite are different from one another.

In various embodiments, the present invention provides a method for converting at least one alcohol to at least one hydrocarbon product, the method comprising: providing a feedstock comprising at least one alcohol; providing a catalyst mixture; contacting the feedstock comprising at least one alcohol with the catalyst mixture to form at least one hydrocarbon product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for converting at least one alcohol to at least one hydrocarbon product, the method comprising: providing at least one alcohol; providing a catalyst mixture; contacting the at least one alcohol with the catalyst mixture to form at least one hydrocarbon product, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a method for converting at least one alcohol to at least one hydrocarbon product, the method comprising: providing a feedstock comprising at least one alcohol; providing a catalyst mixture; contacting the feedstock comprising at least one alcohol with the catalyst mixture to form at least one hydrocarbon product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a method for converting at least one alcohol to at least one hydrocarbon product, the method comprising: providing at least one alcohol; providing a catalyst mixture; contacting the at least one alcohol with the catalyst mixture to form at least one hydrocarbon product, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In some embodiments, the catalyst mixture is a zeolite mixture. In some embodiments, the zeolite mixture comprises at least one first zeolite and at least one second zeolite.

In some embodiments, contacting the feedstock comprising at least one alcohol with the catalyst mixture to form at least one product is performed under conditions effective to form the at least one product.

In some embodiments, contacting the feedstock comprising at least one alcohol with the catalyst mixture to form at least one hydrocarbon product is performed under conditions effective to form the at least one hydrocarbon product.

In some embodiments, contacting the at least one alcohol with the catalyst mixture to form at least one product is performed under conditions effective to form the at least one product.

In some embodiments, contacting the at least one alcohol with the catalyst mixture to form at least one hydrocarbon product is performed under conditions effective to form the at least one hydrocarbon product.

In some embodiments, contacting the feedstock comprising at least one alcohol with the catalyst mixture is performed at a temperature of 180° C. to 600° C.

In some embodiments, contacting the feedstock comprising at least one alcohol with the catalyst mixture is performed at a weight-hourly space velocity (WHSV) of 2 h⁻¹ to 20 h⁻¹.

In some embodiments, the first zeolite comprises at least one first extra-framework species, with the proviso that the at least one first extra-framework species does not comprise aluminum.

In some embodiments, the at least one first extra-framework species is Ga₂O₃.

In some embodiments, the first framework type is MWW, CHA, or MFL

In some embodiments, the first zeolite is Ga-MCM-22 zeolite, Ga-SSZ-13 zeolite, or Ga-ZSM-5 zeolite.

In some embodiments, the first zeolite is Ga-MCM-22 zeolite and the first framework type is MWW, or wherein the first zeolite is a Ga-SSZ-13 zeolite and the first framework type is CHA, or wherein the first zeolite is a Ga-ZSM-5 zeolite and the first framework type is MFI.

In some embodiments, the at least one alcohol is selected from the group consisting of methanol, ethanol, and combination thereof.

In some embodiments, the at least one product is at least one hydrocarbon.

In some embodiments, the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, or any combination thereof.

In some embodiments, the at least one hydrocarbon is at least one olefin, or at least one aromatic hydrocarbon, or both at least one olefin and at least one aromatic hydrocarbon.

In some embodiments, the at least one olefin is selected from the group consisting of ethylene, propylene, and combination thereof.

In some embodiments, the second zeolite is an aluminosilicate zeolite, and wherein the aluminosilicate zeolite comprises a second framework type.

In some embodiments, the second framework type is MWW, CHA, or MFI.

In some embodiments, the aluminosilicate zeolite is Al-MCM-22 zeolite, Al-SSZ-13 zeolite, or Al-ZSM-5 zeolite.

In some embodiments, the aluminosilicate zeolite is Al-MCM-22 zeolite and the second framework type is MWW, or wherein the aluminosilicate zeolite is Al-SSZ-13 zeolite and the second framework type is CHA, or wherein the aluminosilicate zeolite is Al-ZSM-5 zeolite and the framework type is MFI.

In some embodiments, the first zeolite and the second zeolite are in physical contact with one another. In some embodiments, the first zeolite and the second zeolite are not separated from one another.

Non-limiting embodiments include those listed below.

In various embodiments, the present invention provides a system for the dehydration of at least one alcohol, comprising: an inlet port; a reaction chamber, wherein the reaction chamber is in communication with the inlet port, wherein the reaction chamber contains a catalyst mixture, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a system for the dehydration of at least one alcohol, comprising: an inlet port; a reaction chamber, wherein the reaction chamber is in communication with the inlet port, wherein the reaction chamber contains a catalyst mixture, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In various embodiments, the present invention provides a system for converting at least one alcohol to at least one hydrocarbon product, comprising: an inlet port; a reaction chamber, wherein the reaction chamber is in communication with the inlet port, wherein the reaction chamber contains a catalyst mixture, wherein the catalyst mixture comprises at least one first zeolite and at least one second zeolite, wherein the at least one first zeolite is a zeolite of the present invention; and wherein the at least one first zeolite and the at least one second zeolite are different from one another.

In various embodiments, the present invention provides a system for converting at least one alcohol to at least one hydrocarbon product, comprising: an inlet port; a reaction chamber, wherein the reaction chamber is in communication with the inlet port, wherein the reaction chamber contains a catalyst mixture, wherein the catalyst mixture comprises at least one first zeolite and at least one second zeolite, wherein the at least one first zeolite comprises a first microporous framework, wherein the first microporous framework comprises a first framework type, and wherein the first microporous framework comprises silicon atoms, oxygen atoms, and gallium atoms, with the proviso that the first microporous framework does not comprise aluminum atoms; and wherein the at least one first zeolite and the at least one second zeolite are different from one another.

In some embodiments, the first zeolite comprises at least one first extra-framework species, with the proviso that the at least one first extra-framework species does not comprise aluminum.

In some embodiments, the at least one first extra-framework species is Ga₂O₃.

In some embodiments, the first framework type is MWW, CHA, or MFI.

In some embodiments, the first zeolite is Ga-MCM-22 zeolite, Ga-SSZ-13 zeolite, or Ga-ZSM-5 zeolite.

In some embodiments, the first zeolite is Ga-MCM-22 zeolite, Ga-SSZ-13 zeolite, or Ga-ZSM-5 zeolite.

In some embodiments, the first zeolite is Ga-MCM-22 zeolite and the first framework type is MWW.

In some embodiments, the first zeolite is a Ga-SSZ-13 zeolite and the first framework type is CHA.

In some embodiments, the first zeolite is a Ga-ZSM-5 zeolite and the first framework type is MFI.

In some embodiments, the reaction chamber comprises a heater, or a cooler, or both a heater and a cooler.

In some embodiments, the reaction chamber is configured to heat the catalyst mixture to a temperature of 180° C. to 600° C.

In some embodiments, the reaction chamber is configured to contact the feedstock comprising at least one alcohol with the catalyst mixture at a weight-hourly space velocity (WHSV) of 2 h⁻¹ to 20 h⁻¹. In some embodiments, the reaction chamber is configured to contact the at least one alcohol with the catalyst mixture at a weight-hourly space velocity (WHSV) of 2 h⁻¹ to 20 h⁻¹.

In some embodiments, the second zeolite is an aluminosilicate zeolite, and wherein the aluminosilicate zeolite comprises a second framework type.

In some embodiments, the second framework type is MWW, CHA, or MFI.

In some embodiments, the aluminosilicate zeolite is Al-MCM-22 zeolite, Al-SSZ-13 zeolite, or Al-ZSM-S zeolite.

In some embodiments, the aluminosilicate zeolite is Al-MCM-22 zeolite and the second framework type is MWW.

In some embodiments, the aluminosilicate zeolite is Al-SSZ-13 zeolite and the second framework type is CHA.

In some embodiments, the aluminosilicate zeolite is Al-ZSM-5 zeolite and the framework type is MFI.

Non-limiting embodiments include those listed below.

In some embodiments, contacting the feedstock comprising at least one alcohol with the first zeolite is performed at a temperature of 180° C. to 600° C. In some embodiments, contacting the at least one alcohol with the first zeolite is performed at a temperature of 180° C. to 600° C. In some embodiments the temperature is 180° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, contacting the at least one intermediate compound with the second zeolite is performed at a temperature of 180° C. to 600° C. In some embodiments the temperature is 180° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, the first reaction chamber is configured to heat the first zeolite to a temperature of 180° C. to 600° C. In some embodiments the temperature is 180 ° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, the second reaction chamber is configured to heat the second zeolite to a temperature of 180° C. to 600° C. In some embodiments the temperature is 180° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, contacting the feedstock comprising at least one alcohol with the at least one zeolite is performed at a temperature of 180° C. to 600° C. In some embodiments, contacting the at least one alcohol with the at least one zeolite is performed at a temperature of 180° C. to 600° C. In some embodiments the temperature is 180° C. to 600° C., 180° C. to 590° C., 180° C. to 580° C., 180° C. to 570° C., 180° C. to 560° C., 180° C. to SS0° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, the reaction chamber is configured to heat the at least one zeolite to a temperature of 180° C. to 600° C. In some embodiments the temperature is 180° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, contacting the feedstock comprising at least one alcohol with the catalyst mixture is performed at a temperature of 180° C. to 600° C. In some embodiments, contacting the at least one alcohol with the catalyst mixture is performed at a temperature of 180° C. to 600° C. In some embodiments the temperature is 180° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, the reaction chamber is configured to heat the catalyst mixture to a temperature of 180° C. to 600° C. In some embodiments the temperature is 180° C. to 600° C., 180° C. to 590° C., 180° C. to 580° C., 180° C. to 570° C., 180° C. to 560° C., 180° C. to 550° C., 180° C. to 540° C., 180° C. to 530° C., 180° C. to 520° C., 180° C. to 510° C., 180° C. to 500° C., 180° C. to 490° C., 180° C. to 480° C., 180° C. to 470° C., 180° C. to 460° C., 180° C. to 450° C., 180° C. to 440° C., 180° C. to 430° C., 180° C. to 420° C., 180° C. to 410° C., 180° C. to 400° C., 180° C. to 390° C., 180° C. to 380° C., 180° C. to 370° C., 180° C. to 360° C., 180° C. to 350° C., 180° C. to 340° C., 180° C. to 330° C., 180° C. to 320° C., 180° C. to 310° C., 180° C. to 300° C., 180° C. to 290° C., 180° C. to 280° C., 180° C. to 270° C., 180° C. to 260° C., 180° C. to 250° C., 180° C. to 240° C., 180° C. to 230° C., 180° C. to 220° C., 180° C. to 210° C., 180° C. to 200° C., or 180° C. to 190° C. In some embodiments the temperature is 180° C. to 600° C., 190° C. to 600° C., 200° C. to 600° C., 210° C. to 600° C., 220° C. to 600° C., 230° C. to 600° C., 240° C. to 600° C., 250° C. to 600° C., 260° C. to 600° C., 270° C. to 600° C., 280° C. to 600° C., 290° C. to 600° C., 300° C. to 600° C., 310° C. to 600° C., 320° C. to 600° C., 330° C. to 600° C., 340° C. to 600° C., 350° C. to 600° C., 360° C. to 600° C., 370° C. to 600° C., 380° C. to 600° C., 390° C. to 600° C., 400° C. to 600° C., 410° C. to 600° C., 420° C. to 600° C., 430° C. to 600° C., 440° C. to 600° C., 450° C. to 600° C., 460° C. to 600° C., 470° C. to 600° C., 480° C. to 600° C., 490° C. to 600° C., 500° C. to 600° C., 510° C. to 600° C., 520° C. to 600° C., 530° C. to 600° C., 540° C. to 600° C., 550° C. to 600° C., 560° C. to 600° C., 570° C. to 600° C., 580° C. to 600° C., or 590° C. to 600° C.

In some embodiments, contacting the feedstock comprising at least one alcohol with the first zeolite is performed at a weight-hourly space velocity (WHSV) of 2 h⁻¹ to 20 h⁻¹. In some embodiments, contacting the at least one alcohol with the first zeolite is performed at a weight-hourly space velocity (WHSV) of 2 h⁻¹ to 20 h⁻¹. In some embodiments the weight-hourly space velocity (WHSV) is 2 h⁻¹ to 20 h⁻¹, 2 h⁻¹ to 19 h⁻¹, 2 h⁻¹ to 18 h⁻¹, 2 h⁻¹ to 17 h⁻¹, 2 h⁻¹ to 16 h⁻¹, 2 h⁻¹ to 15 h⁻¹, 2 h⁻¹ to 14 h⁻¹, 2 h⁻¹ to 13 h⁻¹, 2 h⁻¹ to 12 h⁻¹, 2 h⁻¹ to 11 h⁻¹, 2 h⁻¹ to 10 h⁻¹, 2 h⁻¹ to 9 h⁻¹, 2 h⁻¹ to 8 h⁻¹, 2 h⁻¹ to 7 h⁻¹, 2 h⁻¹ to 6 h⁻¹, 2 h⁻¹ to 5 h⁻¹, 2 h⁻¹ to 4 h⁻¹, or 2 h⁻¹ to 3 h⁻¹. In some embodiments the weight-hourly space velocity (WHSV) is 2 h⁻¹ to 20 h⁻¹, 3 h⁻¹ to 20 h⁻¹, 4 h⁻¹ to 20 h⁻¹, 5 h⁻¹ to 20 h⁻¹, 6 h⁻¹ to 20 h⁻¹, 7 h⁻¹ to 20 h⁻¹, 8 h⁻¹ to 20 h⁻¹, 9 h⁻¹ to 20 h⁻¹, 10 h⁻¹ to 20 h⁻¹, 11 h⁻¹ to 20 h⁻¹, 12 h⁻¹ to 20 h⁻¹, 13 h⁻¹ to 20 h⁻¹, 14 h⁻¹ to 20 h⁻¹, 15 h⁻¹ to 20 h⁻¹, 16 h⁻¹ to 20 h⁻¹, 17 h⁻¹ to 20 h⁻¹, 18 h⁻¹ to 20 h⁻¹, or 19 h⁻¹ to 20 h⁻¹.

In some embodiments, contacting the at least one intermediate compound with the second zeolite is performed at a weight-hourly space velocity (WHSV) of 2 h⁻¹ to 20 h⁻¹. In some embodiments the weight-hourly space velocity (WHSV) is 2 h⁻¹ to 20 h⁻¹, 2 h⁻¹ to 19 h⁻¹, 2 h⁻¹ to 18 h⁻¹, 2 h⁻¹ to 17 h⁻¹, 2 h⁻¹ to 16 h⁻¹, 2 h⁻¹ to 15 h⁻¹, 2 h⁻¹ to 14 h⁻¹, 2 h⁻¹ to 13 h⁻¹, 2 h⁻¹ to 12 h⁻¹, 2 h⁻¹ to 11 h⁻¹, 2 h⁻¹ to 10 h⁻¹, 2 h⁻¹ to 9 h⁻¹, 2 h⁻¹ to 8 h⁻¹, 2 h⁻¹ to 7 h⁻¹, 2 h⁻¹ to 6 h⁻¹, 2 h⁻¹ to 5 h⁻¹, 2 h⁻¹ to 4 h⁻¹, or 2 h⁻¹ to 3 h⁻¹. In some embodiments the weight-hourly space velocity (WHSV) is 2 h⁻¹ to 20 h⁻¹, 3 h⁻¹ to 20 h⁻¹, 4 h⁻¹ to 20 h⁻¹, 5 h⁻¹ to 20 h⁻¹, 6 h⁻¹ to 20 h⁻¹, 7 h⁻¹ to 20 h⁻¹, 8 h⁻¹ to 20 h⁻¹, 9 h⁻¹ to 20 h⁻¹, 10 h⁻¹ to 20 h⁻¹, 11 h⁻¹ to 20 h⁻¹, 12 h⁻¹ to 20 h⁻¹, 13 h⁻¹ to 20 h⁻¹, 14 h⁻¹ to 20 h⁻¹, 15 h⁻¹ to 20 h⁻¹, 16 h⁻¹ to 20 h⁻¹, 17 h⁻¹ to 20 h⁻¹, 18 h⁻¹ to 20 h⁻¹, or 19 h⁻¹ to 20 h⁻¹.

In some embodiments, contacting the feedstock comprising at least one alcohol with the at least one zeolite is performed at a weight-hourly space velocity (WHSV) of 2 h⁻¹ to 20 h⁻¹. In some embodiments, contacting the at least one alcohol with the at least one zeolite is performed at a weight-hourly space velocity (WHSV) of 2 h⁻¹ to 20 h⁻¹. In some embodiments the weight-hourly space velocity (WHSV) is 2 h⁻¹ to 20 h⁻¹, 2 h⁻¹ to 19 h⁻¹, 2 h⁻¹ to 18 h⁻¹, 2 h⁻¹ to 17 h⁻¹, 2 h⁻¹ to 16 h⁻¹, 2 h⁻¹ to 15 h⁻¹, 2 h⁻¹ to 14 h⁻¹, 2 h⁻¹ to 13 h⁻¹, 2 h⁻¹ to 12 h⁻¹, 2 h⁻¹ to 11 h⁻¹, 2 h⁻¹ to 10 h⁻¹, 2 h⁻¹ to 9 h⁻¹, 2 h⁻¹ to 8 h⁻¹, 2 h⁻¹ to 7 h⁻¹, 2 h⁻¹ to 6 h⁻¹, 2 h⁻¹ to 5 h⁻¹, 2 h⁻¹ to 4 h⁻¹, or 2 h⁻¹ to 3 h⁻¹. In some embodiments the weight-hourly space velocity (WHSV) is 2 h⁻¹ to 20 h⁻¹, 3 h⁻¹ to 20 h⁻¹, 4 h⁻¹ to 20 h⁻¹, 5 h⁻¹ to 20 h⁻¹, 6 h⁻¹ to 20 h⁻¹, 7 h⁻¹ to 20 h⁻¹, 8 h⁻¹ to 20 h⁻¹, 9 h⁻¹ to 20 h⁻¹, 10 h⁻¹ to 20 h⁻¹, 11 h⁻¹ to 20 h⁻¹, 12 h⁻¹ to 20 h⁻¹, 13 h⁻¹ to 20 h⁻¹, 14 h⁻¹ to 20 h⁻¹, 15 h⁻¹ to 20 h⁻¹, 16 h⁻¹ to 20 h⁻¹, 17 h⁻¹ to 20 h⁻¹, 18 h⁻¹ to 20 h⁻¹, or 19 h⁻¹ to 20 h⁻¹.

In some embodiments, contacting the feedstock comprising at least one alcohol with the catalyst mixture is performed at a weight-hourly space velocity (WHSV) of 2 h⁻¹ to 20 h⁻¹. In some embodiments, contacting the at least one alcohol with the catalyst mixture is performed at a weight-hourly space velocity (WHSV) of 2 h⁻¹ to 20 h⁻¹. In some embodiments the weight-hourly space velocity (WHSV) is 2 h⁻¹ to 20 h⁻¹, 2 h⁻¹ to 19 h⁻¹, 2 h⁻¹ to 18 h⁻¹, 2 h⁻¹ to 17 h⁻¹, 2 h⁻¹ to 16 h⁻¹, 2 h⁻¹ to 15 h⁻¹, 2 h⁻¹ to 14 h⁻¹, 2 h⁻¹ to 13 h⁻¹, 2 h⁻¹ to 12 h⁻¹, 2 h⁻¹ to 11 h⁻¹, 2 h⁻¹ to 10 h⁻¹, 2 h⁻¹ to 9 h⁻¹, 2 h⁻¹ to 8 h⁻¹, 2 h⁻¹ to 7 h⁻¹, 2 h⁻¹ to 6 h⁻¹, 2 h⁻¹ to 5 h⁻¹, 2 h⁻¹ to 4 h⁻¹, or 2 h⁻¹ to 3 h⁻¹. In some embodiments the weight-hourly space velocity (WHSV) is 2 h⁻¹ to 20 h⁻¹, 3 h⁻¹ to 20 h⁻¹, 4 h⁻¹ to 20 h⁻¹, 5 h⁻¹ to 20 h⁻¹, 6 h⁻¹ to 20 h⁻¹, 7 h⁻¹ to 20 h⁻¹, 8 h⁻¹ to 20 h⁻¹, 9 h⁻¹ to 20 h⁻¹, 10 h⁻¹ to 20 h⁻¹, 11 h⁻¹ to 20 h⁻¹, 12 h⁻¹ to 20 h⁻¹, 13 h⁻¹ to 20 h⁻¹, 14 h⁻¹ to 20 h⁻¹, 15 h⁻¹ to 20 h⁻¹, 16 h⁻¹ to 20 h⁻¹, 17 h⁻¹ to 20 h⁻¹, 18 h⁻¹ to 20 h⁻¹, or 19 h⁻¹ to 20 h⁻¹.

Non-limiting embodiments include those listed below.

Embodiment 169. A method for the dehydration of at least one alcohol, the method comprising: contacting a feedstock comprising at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

Embodiment 170. The method of embodiment 169, wherein contacting the feedstock comprising at least one alcohol with the at least one zeolite is performed at a temperature of 180° C. to 600° C.

Embodiment 171. The method of embodiment 169, wherein contacting the feedstock comprising at least one alcohol with the at least one zeolite is performed at a weight-hourly space velocity (WHSV) of 2 h⁻¹ to 20 h⁻¹.

Embodiment 172. The method of embodiment 169, wherein the at least one zeolite comprises at least one extra-framework species, with the proviso that the at least one extra-framework species does not comprise aluminum.

Embodiment 173. The method of embodiment 172, wherein the at least one extra-framework species is Ga₂O₃.

Embodiment 174. The method of embodiment 169, wherein the framework type is MWW, CHA, or MFL

Embodiment 175. The method of embodiment 169, wherein the at least one zeolite is Ga-MCM-22 zeolite, Ga-SSZ-13 zeolite, or Ga-ZSM-5 zeolite.

Embodiment 176. The method of embodiment 169, wherein the at least one zeolite is Ga-MCM-22 zeolite and the first framework type is MWW.

Embodiment 177. The method of embodiment 169, wherein the at least one zeolite is a Ga-SSZ-13 zeolite and the first framework type is CHA.

Embodiment 178. The method of embodiment 169, wherein the at least one zeolite is a Ga-ZSM-5 zeolite and the first framework type is MFI.

Embodiment 179. The method of embodiment 169, wherein the at least one alcohol is selected from the group consisting of methanol, ethanol, and combination thereof.

Embodiment 180. The method of embodiment 169, wherein the at least one product is at least one hydrocarbon, dimethyl ether, water, or any combination thereof.

Embodiment 181. The method of embodiment 180, wherein the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, and any combination thereof.

Embodiment 182. The method of embodiment 180, wherein the at least one hydrocarbon is at least one olefin, or at least one aromatic hydrocarbon, or both at least one olefin and at least one aromatic hydrocarbon.

Embodiment 183. The method of embodiment 182, wherein the at least one olefin is selected from the group consisting of ethylene, propylene, and combination thereof.

Embodiment 184. A system for the dehydration of at least one alcohol, comprising: an inlet port; a reaction chamber, wherein the reaction chamber is in communication with the inlet port, wherein the reaction chamber contains at least one zeolite, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms; and an outlet port, wherein the outlet port is in communication with the reaction chamber.

Embodiment 185. The system of embodiment 184, wherein the at least one zeolite comprises at least one extra-framework species, with the proviso that the at least one extra-framework species does not comprise aluminum.

Embodiment 186. The system of embodiment 184, wherein the framework type is MWW, CHA, or MFI.

Embodiment 187. The system of embodiment 184, wherein the at least one zeolite is Ga-MCM-22 zeolite, Ga-SSZ-13 zeolite, or Ga-ZSM-5 zeolite.

Embodiment 188. The system of embodiment 184, wherein the at least one zeolite is Ga-MCM-22 zeolite and the framework type is MWW, or wherein the at least one zeolite is a Ga-SSZ-13 zeolite and the framework type is CHA, or wherein the at least one zeolite is a Ga-ZSM-5 zeolite and the first framework type is MFI.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Experimental Methods

### Materials.

The following chemicals were purchased from Sigma Aldrich: Sodium aluminate (NaAlO₂, technical grade), gallium nitrate (Ga(NO₃)₃, 99.9%), gallium oxide (Ga₂O₃, 99%), hexamethylenimine (HMI, 99%), sodium hydroxide (NaOH, 50 wt %), sodium hydroxide (NaOH, 1M), ammonium nitrate (NH₄NO₃, 99.5%), fumed silica (Cab-o-Sil, M-5, scintillation grade), and tetraethyl orthosilicate (TEOS, 98%). Tetrapropylammonium hydroxide (TPAOH, 40%) was purchased from Thermo Fischer and trimethyladamantylammonium hydroxide (TMAdaOH, 25%) was purchased from SACHEM Inc. Deionized (DI) water was produced using an Aqua Solution RODI-C-12A purification system (18.2 MΩ). All reagents were used as received without further purification.

### Catalyst Preparation.

Aluminosilicate forms of zeolites (Al-zeolites) MCM-22, SSZ-13, and ZSM-5 were synthesized following procedures reported in literature (Parmar, D.; Cha, S. H.; Huang, C.; Chiang, H.; Washburn, S.; Grabow, L. C.; Rimer, J. D., Impact of medium-pore zeolite topology on para-xylene production from toluene alkylation with methanol. Catal. Sci. Technol. 2023, 13 (18), 5227-5236; Dai, W.; Sun, X.; Tang, B., Wu, G., Li, L., Guan, N.; Hunger, M., Verifying the mechanism of the ethene-to-propene conversion on zeolite H-SSZ-13. J. Catal. 2014, 314, 10-20; Perez-Uriarte, P.; Ateka, A.; Gamero, M., Aguayo, A. T.; Bilbao, J., Effect of the Operating Conditions in the Transformation of DME to olefins over a HZSM-5 Zeolite Catalyst. Ind. Eng. Chem. Res. 2016, 55 (23), 6569-6578). The Si/Al molar ratios of Al-zeolite growth mixtures were fixed at 15, 20, and 50, respectively. Gallosilicate isostructures of each zeolite (Ga-zeolites) were synthesized according to the procedures outlined below. The Si/Ga molar ratio of all Ga-zeolite growth mixtures was fixed at 15.

### Synthesis of Ga-MCM-22.

The gallosilicate Ga-MCM-22 (MWW framework type) was synthesized under hydrothermal conditions using a growth mixture with a molar composition of 1 SiO₂: 0.5 HMI: 4 NaOH: 0.033 Ga₂O₃: 35 H₂O. In a typical synthesis, gallium nitrate (0.257 g, 1.00 mmol) was dissolved in a mixture of 1M sodium hydroxide (2.422 g, 60.25 mmol) and DI water (9.550 g, 0.530 mol) while stirring at room temperature. The organic structure-direction agent (OSDA) hexamethylenimine (0.748 g, 7.46 mmol) was then added dropwise to the growth solution, which was then allowed to age overnight at room temperature until homogeneous. Next, fumed silica (0.900 g, 14.97 mmol) was added in small portions to ensure maximum dispersion of the silicon source. The resulting gel was introduced into a 60 ml Teflon liner within a stainless-steel acid digestion bomb (Parr Instruments), which was heated at 150° C. for 7 days in a Thermo Fisher gravity oven at autogenous pressure and under rotation (60 rpm). The vessel was quenched in water to room temperature. The solids were isolated and washed with DI water by centrifugation (6000 rpm) for 5 min three times before drying at 100° C. overnight. As-synthesized Ga-MCM-22 zeolite was calcined in air at 550° C. for 8 h to remove occluded OSDA. The calcined material was then converted to proton form (H-form) by refluxing three times in 1.0 M NH₄NO₃ solution (1.0 g of zeolite per 50 mL of solution) at 80° C. for 2 h followed by calcination at 550° C. for 5 h in a Thermo Scientific Linderberg Blue M box furnace.

### Synthesis of Ga-SSZ-13.

The gallosilicate Ga-SSZ-13 (CHA framework type) was synthesized under hydrothermal conditions using a growth mixture with a molar composition of 1 SiO₂: 0.2 TMAdaOH: 0.2 NaOH: 0.033 Ga₂O₃: 44 H₂O. In a typical synthesis, gallium nitrate hydrate (0.6242 g, 2.42 mmol) was dissolved in a mixture of 50 wt % NaOH (0.593 g, 7.41 mmol), 25 wt % TMAdaOH (6.262 g, 7.41 mmol), and DI water (24.254 g, 1.35 mol). Fumed silica (2.222 g, 36.24 mmol) was then added in small portions to ensure maximum dispersion of the silicon source. The growth mixture was aged overnight under continuous stirring at room temperature. The resulting gel was placed in a Teflon-lined stainless-steel acid digestion bomb and heated at 160° C. for 11 days under static condition in an oven. The resulting solids were washed, dried, and calcined to produce H-form Ga-SSZ-13 using the same procedure described above.

### Synthesis of Ga-ZSM-5.

The gallosilicate Ga-ZSM-5 (MFI framework type) was synthesized under hydrothermal conditions using a growth mixture with a molar composition of 1 SiO₂: 0.15 TPAOH: 0.04 NaOH: 0.033 Ga₂O₃: 25 H₂O. In a typical synthesis, gallium nitrate (0.589 g, 2.30 mmol) was dissolved in a mixture of 50 wt % sodium hydroxide (0.110 g, 1.37 mmol) and DI water (13.73 g, 0.762 mol) while stirring at room temperature. The OSDA tetrapropylammonim hydroxide (2.615 g, 5.14 mmol) and silicon source tetraethyl orthosilicate (7.291 g, 0.034 mol) were added to the growth solution. The solution was allowed to age for 2 h until homogeneous. The resulting gel was introduced into a 60 ml Teflon liner within a stainless-steel acid digestion bomb, which was rotated (60 rpm) at 170° C. and autogenous pressure for 2 days in an oven. Sample isolation and conversion to H-form Ga-ZSM-5 were performed using the same procedure described above.

### Catalyst Characterization.

The crystallinity and phase purity of as-synthesized zeolites were assessed by powder X-ray diffraction (PXRD) using a Rigaku diffractometer (Cu Kα radiation). Textural analysis using the BET method was carried out by obtaining N₂ adsorption and desorption isotherms on a Micrometrics ASAP 2020 instrument. The distribution of Brønsted and Lewis acid sites in H-form zeolites was characterized by Fourier transform infrared (FTIR) spectroscopy using pyridine as the probe molecule at elevated temperatures. Pyridine adsorption-desorption measurements were conducted on self-supporting zeolite wafers (approx. 20 mg, 1.3 cm diameter), activated under dry N₂ at 500° C. for 2 h. Each zeolite wafer was exposed to pyridine-loaded flow of N₂ at 200° C. for 2 h inside an in-house built infrared cell equipped with CaF₂ windows. Subsequent desorption took place at 300° C. for 2 h, and the FTIR spectra were recorded on a Nicolet 6700 spectrometer. Brønsted and Lewis acid site concentrations were determined from the intensities of the infrared bands (1550 and 1450 cm⁻¹, respectively) using extinction coefficients provided by Emeis (Emeis, C., Determination of integrated molar extinction coefficients for infrared absorption bands of pyridine adsorbed on solid acid catalysts. J. Catal. 1993, 141 (2), 347-354). Total acid site concentrations were measured via NH₃ temperature programmed desorption (TPD). Pellets of H-form catalysts were pretreated under 30 sccm of argon (Ar) flow at 550° C. for 5 h. Subsequently, NH₃ adsorption was conducted at 150° C. under flow of 3 scem NH₃ with 30 sccm of Ar serving as the carrier gas for 30 min. Physiosorbed ammonia was removed by flushing the catalyst bed with Ar for 3 h at 150° C. Finally, ammonia desorption occurred by ramping the temperature to 700° C. at a rate of 5° C. min⁻¹. The desorbed ammonia was measured using a Cirrus 3 atmospheric gas analyzer equipped with a quadrupole mass spectrometer. Transmission electron microscopy (TEM) images were obtained using a JEOL 2100 instrument operated at 200 kV in bright field. Powders were mixed with ethanol and sonicated for 5 min prior to be being transferred to a lacey carbon Cu grid mesh 200. Scanning electron microscopy (SEM) was conducted at the Methodist Hospital Research Institute in the Department of Nanomedicine SEM Core using a Nova NanoSEM 230 instrument operated at high vacuum and equipped with a field emission scope providing a high resolution immersion lens. The Nova NanoSEM 230 was also configured with a Bruker SDD-EDS detector for elemental analysis. Powder samples were dispersed in ethanol and sonicated for 5 min prior to being transferred to carbon taped aluminum holders. Sample were also coated with 15 nm of gold to reduce charging effects. Solid-state magic angle spinning (MAS) ⁷¹Ga NMR was performed on a JEOL ECA-500 spectrometer at 11.7 T equipped with a 3.2 mm MAS probe. X-ray photoelectric spectroscopy (XPS) was performed using a PHI 5700 instrument equipped with a monochromatic Al KaX-ray source (1486.6 eV) operated at 350W.

### Catalytic Reactions.

Single and dual bed reactions were carried out in a stainless-steel fixed bed micro-reactor with an inner diameter of 7 mm under atmospheric pressure. Prior to the catalytic experiment, the Ga- or Al-zeolite catalyst was pre-treated under a continuous stream of N₂ (24 sccm) and O₂ (6 sccm) at 550° C. for 6 h and then set to the desired reaction parameters. For alcohol dehydration reactions, we used a temperature of 350° C. and a weight-hourly space velocity (WHSV) of 8 h⁻¹ for methanol transformations and 500° C. and WHSV=19 h⁻¹ for ethanol transformations. For tandem reactions using a dual bed configuration, we used 350° C. and WHSV=5 h⁻¹ for methanol to hydrocarbons (MTH) reactions and 500° C. and WHSV=3 h⁻¹ for ethanol to propylene reactions. In a typical experiment, the upstream bed contained 0.2 g of Ga-zeolite catalyst pellets and 0.6 g silica gel (Davisol Grade 636, 36-60 mesh size); and the downstream bed contained 0.3 g of Al-zeolite catalyst pellets and 0.9 g silica gel. The two beds were separated using quartz wool and WHSV values for each bed are specified in the discussion section. The reactor effluent was analyzed on-line with an Agilent 7890B gas chromatography (GC) equipped with a DB-1 capillary column (0.25 mm×60 m) and a flame ionization detector (FID). Space time yield (STY) calculations of propylene in ethanol to propylene tandem reactions were done according to equation (3).

$$STY = \frac{desired product quantity}{catalyst weight \cdot time} ( μmol {g}^{- 1} {h}^{- 1} ) \qquad ( 3 )$$

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature, or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention is not limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

What is claimed is:

1. A method for the dehydration of at least one alcohol, the method comprising:
contacting a feedstock comprising at least one alcohol with at least one zeolite to form at least one product, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms.

2. The method of claim 1, wherein contacting the feedstock comprising at least one alcohol with the at least one zeolite is performed at a temperature of 180° C. to 600° C.

3. The method of claim 1, wherein contacting the feedstock comprising at least one alcohol with the at least one zeolite is performed at a weight-hourly space velocity (WHSV) of 2 h⁻¹ to 20 h⁻¹.

4. The method of claim 1, wherein the at least one zeolite comprises at least one extra-framework species, with the proviso that the at least one extra-framework species does not comprise aluminum.

5. The method of claim 4, wherein the at least one extra-framework species is Ga₂O₃.

6. The method of claim 1, wherein the framework type is MWW, CHA, or MFI.

7. The method of claim 1, wherein the at least one zeolite is Ga-MCM-22 zeolite, Ga-SSZ-13 zeolite, or Ga-ZSM-5 zeolite.

8. The method of claim 1, wherein the at least one zeolite is Ga-MCM-22 zeolite and the first framework type is MWW.

9. The method of claim 1, wherein the at least one zeolite is a Ga-SSZ-13 zeolite and the first framework type is CHA.

10. The method of claim 1, wherein the at least one zeolite is a Ga-ZSM-5 zeolite and the first framework type is MFI.

11. The method of claim 1, wherein the at least one alcohol is selected from the group consisting of methanol, ethanol, and combination thereof.

12. The method of claim 1, wherein the at least one product is at least one hydrocarbon, dimethyl other, water, or any combination thereof.

13. The method of claim 12, wherein the at least one hydrocarbon is selected from the group consisting of at least one C₁ hydrocarbon, at least one C₂ hydrocarbon, at least one C₃ hydrocarbon, at least one C₄ hydrocarbon, at least one C₅ hydrocarbon, at least one C₆ hydrocarbon, at least one C₇ hydrocarbon, at least one C₈ hydrocarbon, and any combination thereof.

14. The method of claim 12, wherein the at least one hydrocarbon is at least one olefin, or at least one aromatic hydrocarbon, or both at least one olefin and at least one aromatic hydrocarbon.

15. The method of claim 14, wherein the at least one olefin is selected from the group consisting of ethylene, propylene, and combination thereof.

16. A system for the dehydration of at least one alcohol, comprising: an inlet port; a reaction chamber, wherein the reaction chamber is in communication with the inlet port, wherein the reaction chamber contains at least one zeolite, wherein the at least one zeolite comprises a microporous framework, wherein the microporous framework comprises a framework type, and wherein the microporous framework comprises silicon atoms, oxygen atoms, and gallium atoms, with the proviso that the microporous framework does not comprise aluminum atoms; and an outlet port, wherein the outlet port is in communication with the reaction chamber.

17. The system of claim 16, wherein the at least one zeolite comprises at least one extra-framework species, with the proviso that the at least one extra-framework species does not comprise aluminum.

18. The system of claim 16, wherein the framework type is MWW, CHA, or MFI.

19. The system of claim 16, wherein the at least one zeolite is Ga-MCM-22 zeolite, Ga-SSZ-13 zeolite, or Ga-ZSM-5 zeolite.

20. The system of claim 16, wherein the at least one zeolite is Ga-MCM-22 zeolite and the framework type is MWW, or wherein the at least one zeolite is a Ga-SSZ-13 zeolite and the framework type is CHA, or wherein the at least one zeolite is a Ga-ZSM-5 zeolite and the first framework type is MFI.
